(19) United States
(12) Patent Application Publication
(10) Pub. No.: US 2019/0201315 A1
(43) Pub. Date: Jul. 4, 2019

(54) REVERSE WASH CONDITIONING COMPOSITION AND METHODS OF USE

(71) Applicant: L'OREAL, Paris (FR)

(72) Inventors: Halil GEVGILILI, Weehawken, NJ (US); Semra SENTURK-OZER, Westfield, NJ (US)

(21) Appl. No.: 15/859,990

(22) Filed: Jan. 2, 2018

(51) Int. Cl. A61K 8/898; A61K 8/895; A61K 8/81; A61K 8/46; A61K 8/37; A61K 8/34; A61Q 5/12

(52) U.S. Cl. CPC A61K 8/898; A61K 8/895; A61K 8/8158; A61Q 5/12; A61K 8/37; A61K 8/342; A61K 8/463

(57) ABSTRACT

The present disclosure relates to hair conditioning compositions and to methods for conditioning hair using the hair conditioning compositions. The hair conditioning compositions typically include: at least one amino functional silicone; at least one nonionic silicone polymer; at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type; and water. The hair conditioning compositions are unique because they are particularly useful for conditioning hair before the hair is washed with a shampoo. Thus, the present disclosure also relates to methods of conditioning hair and in particular in a routine for conditioning hair where the hair is first treated with a hair conditioning composition followed by treatment with a shampoo.

### FIELD OF THE DISCLOSURE

The present disclosure relates to hair conditioning compositions that can be used before washing the hair with a shampoo, which is the reverse of a typical shampooing and conditioning routine. The disclosure further relates to methods of using the conditioning compositions.

### BACKGROUND

Individuals desire healthy and strong hair, a healthy looking hair is in general a sign of good health and good hair-care practices. Nonetheless, nutrition, environmental influences, and chemical hair treatments can lead to hair damage that significantly weakens and dulls the hair over time. Gloss and moisture balance are deleteriously affected making the hair more difficult to comb and style. Furthermore, dry hair that has been weakened or damages is also prone to breakage and the formation of “split ends.”

Nutrition plays a crucial role in the health of hair, but nutrition alone is not sufficient to compensate for the various types of physical, chemical, and environmental damage that prevent optimal hair quality. Physical hair damage is often the result of repeatedly manipulating the shape of the hair. For example, hair styles such as ponytails, buns, and braiding are quick and easy but when done too often and too tightly, can impart strain on the edges of the hair and cause a receding hair line. Hair also becomes physically damaged during detangling and styling. Excessive detangling can result in split ends and breakage.

Many chemical treatments are available for changing the appearance of hair. For example, hair may be lightened or bleached and oxidative dyes can be used to change the color of the hair. Chemical treatments for permanently straightening or curling the hair are also common. Chemical treatments are popular because their effects are long-lasting and can be drastic. Nonetheless, the biggest drawback to chemical treatments is the strain and damage they cause to the hair. This is because chemical treatments permanently change the chemical and physical structure of the hair. Another cause of chemical hair damage is heat. Repeated use of heating appliances such as flat irons and blow-dryers remove moisture from the surface of the hair cuticles, resulting in brittle, dry hair that become more vulnerable to breakage.

The environment also influences the health of hair. Regions with hard water can affect the look, feel and shine of the hair. This is because hard water leaves mineral deposits, which accumulate over time on the hair and eventually prevents moisture intake into the hair. The hair becomes dry, frizzy, and is prone to tangles. Environmental factors, such as strong sun, wind, cold air, extreme temperature variations and changes in air humidity can also damage the hair. The static and dry winter air contributes to moisture loss. Abrupt change from cold outdoor air to warm indoor air can cause the cuticle layers of the hair to lose moisture quickly into the atmosphere. Environmental effects on the hair, however, cannot be completely avoided. Thus, mechanisms to reduce or prevent damage to hair, and products that can nourish and strengthen hair are useful for combating hair damage.

One other factor that can lead to making the hair feel more damaged, tangled, more dry and less soft/smooth is the cleansing process that involves the use of shampoos. Shampoos contain detergents or surfactants such as anionic surfactants which, while they produce good foams and effectively cleanse the hair, can also contribute to said adverse effects on hair.

In order to improve the integrity, look, and feel of hair, including hair that has become dry or damaged, consumers turn to hair conditioners. Hair conditioners are products that are applied to hair to alter the hair's texture, appearance, shine, etc. Hair conditioners are often viscous liquids that are applied and massaged into the hair typically after the hair has been washed with a shampoo. Conditioners can hydrate, soften, and help to detangle hair. Cationic compounds (compounds that have a cationic counterion or compounds that can be cationically charged depending on the pH of a composition) can be added into conditioners in order to enhance the conditioning properties of such products but cationic compounds are generally easily washed off from the hair, especially upon a consecutive shampooing step. One solution is to include silicones into conditioners because they can provide additional smoothing benefits to hair and contribute to the wash resistance of the conditioners. However, silicone compounds can also weigh the hair down too much and make it more difficult to style the hair after the shampooing-conditioning process. Thus, manufacturers of hair care products continue to seek the right balance of the amount and/or types of ingredients or combinations of ingredients as well as alternative methods of providing hair care benefits to the hair during the cleansing process.

### SUMMARY OF THE DISCLOSURE

The present disclosure relates to conditioning compositions. A unique feature of the conditioning compositions of the present disclosure is that they are particularly effective when applied to the hair before washing the hair with a shampoo. This is the reverse of a typical shampooing and conditioning routine wherein the conditioning step comes after the shampooing step. The hair conditioning compositions of the present disclosure impart conditioning effects that are durable and withstand subsequent washing of the hair with shampoo. The conditioning compositions provide a multitude of benefits such as detangling, combability, softness, frizz control, body, shine, and moisturizing effects to the hair, without the negative drawbacks associated with typical conditioners and conditioning routines. For example, the conditioning compositions and methods of the present disclosure, unlike typical conditioners and routines, do not leave hair having an undesirable coating, heaviness, or a greasy feel.

The conditioning compositions include a synergistic combination of at least two silicones and at least one ampholytic polymer. One of the silicones is an amino functional silicone, for example, a high charge density, amino functional silicone. The other silicone is a nonionic silicone polymer, for example, a high molecular weight nonionic silicone copolymer. The ampholytic polymer typically comprises a repetition of: (i) one or more units obtained from a monomer of (meth)acrylamide type, (ii) one or more units obtained from a monomer of (meth)acrylamidoalkyltrialkyl-ammonium type, and (iii) one or more units obtained from an acidic monomer of (meth)acrylic acid type. Thus, the conditioning compositions of the instant disclosure typically include at least the following combination of components:
at least one amino functional silicone;
at least one high molecular weight nonionic silicone polymer; and
at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type.

The at least one amino functional silicone may be a high molecular weight silicone compound. It may also be, for example, a functionalized amodimethicone. Non-limiting examples include bis(C13-15 alkoxy) PG-amodimethicone.

The at least one nonionic silicone polymer may be particles dispersed in an aqueous dispersion medium. Non-limiting examples of useful nonionic silicone polymers include polymethylsiloxane resin, a linear block copolymer, and a mixture thereof. An example of a particularly useful nonionic silicone polymer is a divinyl-dimethicone/dimethicone copolymer.

As already noted, the at least one ampholytic polymer includes a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type. The ampholytic polymer may have a cationic charge density of greater than 1 meq/g to about 3.5 meq/g.

In some instances, the at least one ampholytic polymer includes units derived from the following monomers: (i) acrylamide, (ii) methacrylamidopropyltrimethyl-ammonium chloride, and (iii) acrylic acid, for example, an ampholytic terpolymer of methacrylamidopropyl-trimethylammonium chloride (MAPTAC), acrylamide, and acrylic acid. Additionally, in some cases, monomer (i) of the at least one ampholytic polymer is methacrylic acid amide, monomer (ii) is N-vinylpyrrolidone, and monomer (iii) is 3-alkyl-1-vinylimidazolium salts with acceptable anions, for example, monomer (iii) may be 3-methyl-1-vinylimidazolium methyl sulfate. In some instances a particularly useful ampholytic polymer is polyquaternium-53.

At least one cationic surfactant may be included in the conditioning compositions. Non-limiting example of cationic surfactants that may be included in the conditioning compositions include cetrimonium chloride, cetrimonium methosulfate, stearimonium chloride, behentrimonium chloride, behentrimonium methosulfate, behenamidopropyltrimonium methosulfate, dipalmitoylethyl hydroxyethylmonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, isostearamidopropyl dimethylamine, oleyl hydroxyethyl imidazoline, stearamidopropyldimethylamine, behenamidopropyl-dimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, arachidamidoethyidimethylamine, quaternium-91, and a mixture thereof.

At least one non-silicone fatty compound may be included in the conditioning compositions. Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. In some instances, the at least one non-silicone fatty compound includes one or more fatty alcohols, fatty acids, esters of fatty acids and fatty alcohols (for example, cetyl palmitate, cetyl stearate, myristyl myristate, myristyl stearate, cetyl myristate, and stearyl stearate).

The conditioning compositions may include additional ingredients, for example, water-soluble solvents, thickening agents, surfactants, including nonionic surfactants, preservatives, pH adjustors, salts, fragrances, etc.

The instant disclosure also relates to methods of using the hair conditioning compositions, as the compositions are useful for conditioning hair, especially hair of the head, and are unique in their ability to impart effects such as detangling, combing, softness that are durable and withstand subsequent washing of the hair with shampoo. Although the conditioning compositions can be used independently of a cleansing/shampooing treatment or can be used after a cleansing/shampooing treatment, the conditioning compositions are particularly well-suited for use immediately before a cleansing/shampooing procedure. Thus, in some cases, the instant disclosure relates to methods for conditioning hair, the method including treating the hair (conditioning the hair) with a conditioning composition of the instant disclosure followed by treating the hair (cleansing the hair) with a shampoo, i.e., the hair conditioning compositions are used in a “reverse-wash” routine.

Finally, the instant disclosure relates to kits comprising the hair conditioning compositions. Such kits may include a hair conditioning composition of the instant disclosure and a shampoo, wherein the hair conditioning composition and the shampoo are separately contained. The hair conditioning composition and the shampoo may be included in separate bottles or containers or may be included in an assembly or unitary device or packaging that separately houses the hair conditioning composition and the shampoo. Instructions, mixing components, brushes, gloves, measuring tools, etc., may also be included in the kits.

### DETAILED DESCRIPTION OF THE DISCLOSURE

The benefits imparted by typical conditioners do not withstand subsequent shampooing and are therefore, are not lasting effects. The present disclosure relates to conditioning compositions (“conditioners”) that are particularly useful in a hair-treatment routine, wherein the hair is treated with the hair conditioning composition before being treated with a shampoo. This is the reverse of a typical shampooing and conditioning routine. Therefore, the conditioning compositions may be referred to as “reverse-wash conditioning compositions” or “reverse-wash conditioners.” Although the conditioning compositions are particularly useful for use before washing the hair with a shampoo, they are also useful independent of a shampoo, or may be used after cleansing the hair with a shampoo. Regardless of the particular routine in which the conditioning compositions are used, unlike typical conditioners and routines, the conditioning compositions of the instant disclosure do not leave hair with an undesirable coating, heaviness, or a greasy feel.

The conditioning compositions include a unique combination of at least two silicones and an ampholytic polymer. One of the silicones is an amino silicone, for example, a high charge density, amino functional silicone. The other silicone is a nonionic silicone polymer, for example, a high molecular weight nonionic silicone copolymer. The ampholytic polymer may also be referred to as an “amphoteric polymer,” and relates to a polymer (including a copolymer) in which ionic groups of the opposite sign are incorporated into the same pendant groups. In particular, useful ampholytic polymers include those comprising a repetition of: (i) one or more units obtained from a monomer of (meth)acrylamide type, (ii) one or more units obtained from a monomer of (meth)acrylamidoalkyltrialkyl-ammonium type, and (iii) one or more units obtained from an acidic monomer of (meth)acrylic acid type.

The conditioning compositions of the instant disclosure typically include:
at least one amino functional silicone, for example, a high charge density terminally modified amino functional silicone;
at least one nonionic silicone polymer, for example, a high molecular weight nonionic silicone copolymer;
at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
optionally, at least one cationic surfactant;
optionally, at least one non-silicone fatty compound; and water.

In one embodiment, the hair conditioning composition may include:

about 0.1 to about 15 wt. %, about 0.1 to about 10 wt. %, about 1 to about 5 wt. %, or about 1 to about 3 wt. %, of at least one amino functional silicone, for example, a functionalized amodimethicone, such as bis(C13-15 alkoxy) PG-amodimethicone, amodimethicone, and a mixture thereof;

about 0.1 to about 15 wt. %, about 0.1 to about 10 wt. %, about 1 to about 5 wt. %, or about 1 to about 3 wt. %, of at least one nonionic silicone polymer, for example, a nonionic polymethylsiloxane resin, a nonionic linear block copolymer, and a mixture thereof (and in particular, divinyl-dimethicone/dimethicone copolymer);

about 0.01 to about 10 wt. %, about 0.05 to about 5 wt. %, or about 0.1 to about 4 wt. %, or 0.3 to about 2 wt. %, of at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type, for example, an ampholytic terpolymer consisting of acrylic acid, methacrylamidopropyl trimethyl ammonium chloride (and in particular, polyquaternium-53);

0.01 to about 10 wt. %, about 0.01 to about 5 wt. %, or about 0.1 to about 5 wt. % of at least one cationic surfactant, for example, cetrimonium chloride, cetrimonium methosulfate, stearimonium chloride, behentrimonium chloride, behentrimonium methosulfate, behenamidopropyltrimonium methosulfate, dipalmitoylethyl hydroxyethylmonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, isostearamidopropyl dimethylamine, oleyl hydroxyethyl imidazoline, stearamidopropyldimethylamine, behenamidopropyl-dimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, arachidamidoethyidimethylamine, quaternium-91, and a mixture thereof (and in particular dipalmitoylethyl hydroxyethylmonium methosulfate, behentrimonium chloride, cetrimomonium chloride, and a mixture thereof);

0.1 to about 20 wt. %, about 0.1 to about 15 wt. %, or about 1 to about 10 wt. % of at least one non-silicone fatty compound, for example, one or more fatty alcohols and/or one or more esters of fatty alcohols;

optionally, about 0.1 to about 30 wt. %, about 0.1 to about 20 wt. %, about 1 to about 15 wt. % of at least one water-soluble solvents, for example, organic solvents such as alcohols (e.g.,C₁₋₄ alcohols), polyols, glycols, and a mixture thereof;

optionally, about 0.01 to about 10, about 0.01 to about 5, or about 0.1 to about 5 wt. % of one or more nonionic surfactants, for example, oxyethylenated fatty alcohols, block-copolymer (polycondensate) surfactants of ethylene oxide and of propylene oxide, and a mixture thereof; and

about 50 to about 95 wt. %, about 70 to about 95 wt. %, or about 75 to about 90 wt. % of water;

wherein all weights being based on the total weight of the composition.

In the above embodiment, the hair conditioning composition may be in the form of an emulsion, for example, an oil-in-water emulsion. Additional components described throughout this disclosure including preservatives, pH adjusters, salts, fragrances, etc., may also be included in the hair conditioning composition.

In another embodiment, the hair conditioning composition may include:

about 0.1 to about 10 wt. % or about 0.1 to about 5 wt. % of BIS(C13-15 alkoxy) PG-amodimethicone;

about 0.1 to about 10 wt. % or about 0.1 to about 5 wt. % of divinyldimethicone/dimethicone copolymer;

about 0.01 to about 5 wt. % of polyquaternium-53;

about 0.01 to about 5 wt. % or about 0.01 to about 5 wt. % of at least one cationic surfactant;

about 1 to about 15 wt. % of at least one non-silicone fatty compound, for example, fatty alcohols and/or one or more esters of fatty alcohols;

optionally, about 0.01 to about 5 or about 0.1 to about 5 wt. % of one or more nonionic surfactants, for example, oxyethylenated fatty alcohols, block-copolymer (polycondensate) surfactants of ethylene oxide and of propylene oxide, and a mixture thereof; and

about 70 to about 95 wt. % or about 75 to about 90 wt. % of water;

wherein all weights being based on the total weight of the composition.

In the above embodiments, the hair conditioning composition may be in the form of an emulsion, for example, an oil-in-water emulsion. Additional components described throughout this disclosure including preservatives, pH adjusters, salts, fragrances, etc., may also be included in the hair conditioning composition.

As already noted, the hair conditioning compositions of the disclosure are useful in methods for conditioning hair. Such methods typically include applying a hair conditioning composition of the instant disclosure to the hair and rinsing the conditioning composition from the hair with water. After rinsing the hair conditioning composition from the hair, the hair may be subsequently cleansed with a shampoo. As is understood in the art, cleansing the hair with a shampoo (or shampooing the hair) relates to applying a cleansing composition to the hair and rinsing the cleansing composition from the hair with water to remove the cleansing composition and the unwanted contaminants, dirt, grease, etc., from the hair.

Finally, the instant disclosure relates to kits comprising the hair conditioning compositions. Such kits may include a hair conditioning composition of the instant disclosure and a shampoo, wherein the hair conditioning composition and the shampoo are separately contained. Instructions, mixing components, brushes, gloves, measuring tools, etc., may also be included in the kits.

More exhaustive but non-limiting lists of components useful in the hair conditioning compositions of the instant disclosure are provided below.

### Amino-Functional Silicone

The term “amino functional silicone” is intended to mean any silicone comprising at least one primary, secondary or tertiary amine or a quaternary ammonium group (i.e., a quaternized group).

As amino functional silicones that may be used in the scope of the instant disclosure, the following can be cited:

a) polysiloxanes corresponding to formula (A):

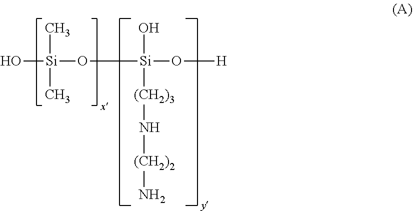

in which x′ and y′ are integers such that the weight-average molecular weight (Mw) is comprised between about 5000 and 500 000;

b) amino silicones corresponding to formula (B):

R′ₐG₃₋ₐ-Si(OSiG₂)ₙ-(OSiG_{b}R′_{2-b})ₘ—O—SiG₃₋ₐ-R′ₐ (B)

in which:

G, which may be identical or different, designate a hydrogen atom, or a phenyl, OH or C₁-C₈ alkyl group, for example methyl, or C₁-C₈ alkoxy, for example methoxy,

a, which may be identical or different, denote the number 0 or an integer from 1 to 3, in particular 0;

b denotes 0 or 1, and in particular 1;

m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;

R′, which may be identical or different, denote a monovalent radical having formula -CqH₂qL in which q is a number ranging from 2 to 8 and L is an optionally quaternized amino group chosen from the following groups:

—NR″-Q-N(R″)₂

—N(R″)₂

—N+(R″)₃A-

—N+H(R″)₂A-

—N+H₂(R″)A-

—N(R″)-Q-N+R″H₂A-

—NR″-Q-N+(R″)₂HA-

—NR″-Q-N+(R″)₃A-,

in which R″, which may be identical or different, denote hydrogen, phenyl, benzyl, or a saturated monovalent hydrocarbon-based radical, for example a C₁-C₂₀ alkyl radical; Q denotes a linear or branched CrH₂ᵣ group, r being an integer ranging from 2 to 6, preferably from 2 to 4; and A- represents a cosmetically acceptable ion, in particular a halide such as fluoride, chloride, bromide or iodide.

A group of amino functional silicones corresponding to this definition (B) is represented by the silicones called “trimethylsilylamodimethicone” having formula (C):

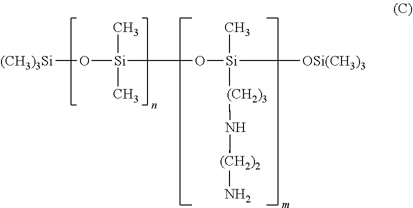

in which n and m have the meanings given above, in formula B.

Another group of amino functional silicones corresponding to this definition is represented by silicones having the following formulae (D) or (E):

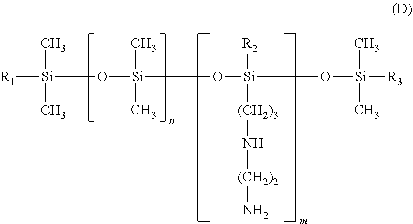

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;
R₁, R₂, R₃, which may be identical or different, represent a hydroxy or C₁-C₄ alkoxy radical, where at least one of the radicals R₁ to R₃ denotes an alkoxy radical.

The alkoxy radical is preferably a methoxy radical.

The hydroxy/alkoxy mole ratio ranges preferably from 0.2:1 to 0.4:1 and preferably from 0.25:1 to 0.35:1 and more particularly equals 0.3:1.

The weight-average molecular weight (Mw) of the silicone ranges preferably from 2000 to 1 000 000, more particularly from 3500 to 200 000.

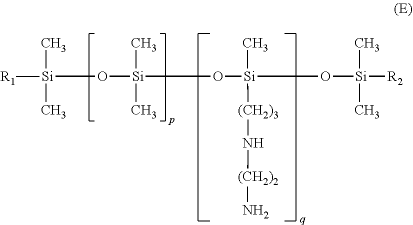

in which:

p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R₁, R₂, which are different, represent a hydroxy or C₁-C₄ alkoxy radical, where at least one of the radicals R₁ or R₂ denotes an alkoxy radical.

The alkoxy radical is preferably a methoxy radical.

The hydroxy/alkoxy mole ratio ranges generally from 1:0.8 to 1:1.1 and preferably from 1:0.9 to 1:1 and more particularly equals 1:0.95.

The weight-average molecular weight (Mw) of the silicone ranges preferably from 2000 to 200 000, even more particularly 5000 to 100 000 and more particularly from 10 000 to 50 000.

Commercial products corresponding to these silicones having structure (D) or (E) may include in their composition one or more other amino silicones whose structure is different than formulae (D) or (E).

A product containing amino silicones having structure (D) is sold by Wacker under the name Belsil® ADM 652.

A product containing amino silicones having structure (E) is sold by Wacker under the name Fluid WR 1300e.

When these amino functional silicones are used, one particularly advantageous embodiment consists in using them in the form of an oil-in-water emulsion. The oil-in-water emulsion may comprise one or more surfactants. The surfactants may be of any nature but are preferably cationic and/or nonionic. The number-average size of the silicone particles in the emulsion generally ranges from 3 nm to 500 nanometres. Preferably, in particular as amino functional silicones having formula (E), microemulsions are used whose average particle size ranges from 5 nm to 60 nanometres (limits included) and more preferably from 10 nm to 50 nanometres (limits included). Accordingly, according to the invention the microemulsions of amino functional silicone having formula (E) sold as Finish CT 96 E® or SLM 28020® by Wacker can be used.

Another group of amino functional silicones corresponding to this definition is represented by the following formula (F):

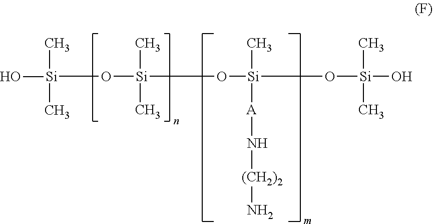

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably linear.

The weight-average molecular weight (Mw) of these amino functional silicones ranges preferably from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino functional silicones corresponding to this definition is represented by the following formula (G):

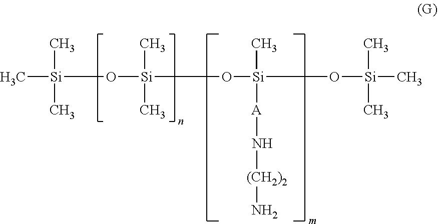

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and preferably 4 carbon atoms. This radical is preferably branched.

The weight-average molecular weight (Mw) of these amino functional silicones ranges preferably from 500 to 1 000 000 and even more particularly from 1000 to 200 000.

A silicone having this formula is for example DC2-8566 Amino Fluid by Dow Corning.

c) amino functional silicones corresponding to formula (H):

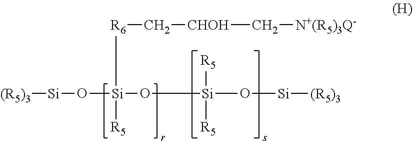

in which:
R₅ represents a monovalent hydrocarbon-based radical containing from 1 to 18 carbon atoms, and in particular a C₁-C₁₈ alkyl or C₂-C₁₈ alkenyl radical, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
Q- is an anion such as a halide ion, in particular chloride, or an organic acid salt (for example acetate);

r represents a mean statistical value from 2 to 20 and in particular from 2 to 8;
s represents a mean statistical value from 20 to 200 and in particular from 20 to 50.

Such amino functional silicones are described more particularly in patent U.S. Pat. No. 4,185,087.

d) quaternary ammonium silicones having formula (I):

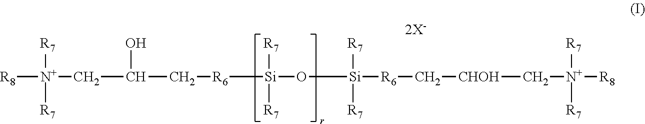

in which:
R₇, which may be identical or different, represent a monovalent hydrocarbon-based radical containing from 1 to 18 carbon atoms, and in particular a C₁-C₁₈ alkyl radical, a C₂-C₁₈ alkenyl radical or a ring containing 5 or 6 carbon atoms, for example methyl;
R₆ represents a divalent hydrocarbon-based radical, in particular a C₁-C₁₈ alkylene radical or a divalent C₁-C₁₈, for example C₁-C₈, alkylenoxy radical linked to the Si via an SiC bond;
R₈, which may be identical or different, represent a hydrogen atom, a monovalent hydrocarbon-based radical containing from 1 to 18 carbon atoms, and in particular a C₁-C₁₈ alkyl radical, a C₂-C₁₈ alkenyl radical or a —R₆—NHCOR₇ radical;
X— is an anion such as a halide ion, in particular chloride, or an organic acid salt (for example acetate);
r represents a mean statistical value from 2 to 200 and in particular from 5 to 100;

These silicones are described, for example, in patent application EP-A 0 530 974.

e) amino functional silicones having formula (J):

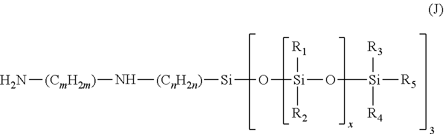

in which:
R₁, R₂, R₃ and R₄, which may be identical or different, denote a C₁-C₄ alkyl radical or a phenyl group;
R₅ denotes a C₁-C₄ alkyl radical or a hydroxyl group;
n is an integer ranging from 1 to 5;
m is an integer ranging from 1 to 5;

and in which x is chosen such that the amine number is between 0.01 and 1 meq/g;

f) multiblockpolyoxyalkylenated amino silicones, of type (AB)n, A being a polysiloxane block and B being a polyoxyalkylenated block containing at least one amine group.

Said silicones are preferably constituted of repeating units having the following general formulae:

[-(SiMe₂O)xSiMe₂—R—N(R″)—R′—O(C₂H₄O)ₐ(C₃H₆O)b-R′—N(H)—R—]

or alternatively

[-(SiMe₂O)xSiMe₂—R—N(R″)—R′—O(C₂H₄O)ₐ(C₃H₆O)b-]

in which:
a is an integer greater than or equal to 1, preferably ranging from 5 to 200, more particularly ranging from 10 to 100;
b is an integer comprised between 0 and 200, preferably ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C₂-C₁₂ hydrocarbon-based radical, optionally including one or more heteroatoms such as oxygen; preferably, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a divalent linear or branched C₂-C₁₂ hydrocarbon-based radical, optionally including one or more heteroatoms such as oxygen; preferably, R¹ denotes an ethylene radical, a linear or branched propylene radical, a linear or branched butylene radical, or a —CH₂CH₂CH₂OCH(OH)CH₂— radical; preferentially R¹ denotes —CH(CH₃)—CH₂—.

The siloxane blocks preferably represent between 50 and 95 mol % of the total weight of the silicone, more particularly from 70 to 85 mol %.

The amine content is preferably between 0.02 and 0.5 meq/g of copolymer in a 30% solution in dipropylene glycol, more particularly between 0.05 and 0.2.

The weight-average molecular weight (Mw) of the silicone is preferably comprised between 5000 and 1 000 000, more particularly between 10 000 and 200 000.

Mention may be made especially of the silicones sold under the names Silsoft™ A-843 or Silsoft™ A+ by Momentive.

g) the alkylamino silicones corresponding to formula (K) below:

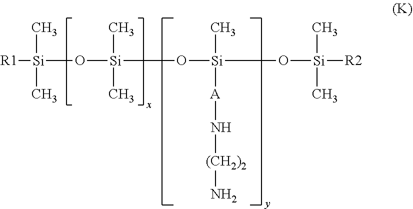

in which:
x and y are numbers ranging from 1 to 5000; preferably, x ranges from 10 to 2000 and especially from 100 to 1000; preferably, y ranges from 1 to 100;
R₁ and R₂, which may be identical or different, preferably identical, are linear or branched, saturated or unsaturated alkyl radicals, comprising 6 to 30 carbon atoms, preferably 8 to 24 carbon atoms and especially 12 to 20 carbon atoms;
A denotes a linear or branched alkylene radical containing from 2 to 8 carbon atoms. Preferably, A comprises 3 to 6 carbon atoms, especially 4 carbon atoms; preferably, A is branched. Mention may be made especially of the following divalent radicals: —CH₂CH₂CH₂ and —CH₂CH(CH₃)CH₂—.

Preferably, R₁ and R₂, which may be identical or different, are saturated linear alkyl radicals comprising 6 to 30 carbon atoms, preferably 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R₁ and R₂, which may be identical or different, are chosen from hexadecyl (cetyl) and octadecyl (stearyl) radicals.

Preferentially, the silicone is of formula (K) with:
x ranging from 10 to 2000 and especially from 100 to 1000;
y ranging from 1 to 100;
A comprising 3 to 6 carbon atoms and especially 4 carbon atoms; preferably, A is branched; and more particularly A is chosen from the following divalent radicals: CH₂CH₂CH₂ and —CH₂CH(CH₃)CH₂—; and
R₁ and R₂, which may be identical or different, being linear, saturated alkyl radicals comprising 6 to 30 carbon atoms, preferably 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R₁ and R₂, which may be identical or different, being chosen from hexadecyl (cetyl) and octadecyl (stearyl) radicals.

An example of silicone of formula (K) is bis-cetearylamodimethicone (INCI name).

Mention may be made especially of the silicone sold under the name Silsoft™ AX by Momentive.

h) silicone compounds with at least one quaternary ammonium group.

Suitable non-limiting examples are quaternium 80, silicone quaternium-1, silicone quaternium-2, silicone quaternium-2 panthenol succinate, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-16, silicone quaternium-16/Glycidoxy Dimethicone Crosspolymer, silicone quaternium-17, silicone quaternium-18, silicone quaternium-20 and silicone quaternium-21. Preferred are quaternium 80, silicone quaternium-16, silicone quaternium-18, silicone quaternium-1, silicone quaternium-2, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, silicone quaternium-17, silicone quaternium-20 and silicone quaternium-21. More preferred are quaternium 80, silicone quaternium-16, silicone quaternium-18, silicone quaternium-3, silicone quaternium-4, silicone quaternium-5, silicone quaternium-6, silicone quaternium-7, silicone quaternium-8, silicone quaternium-9, silicone quaternium-10, silicone quaternium-11, silicone quaternium-12, silicone quaternium-15, and silicone quaternium-17. Most preferred are quaternium 80, silicone quaternium-16, silicone quaternium-18, silicone quaternium-15, and mixtures thereof.

The silicone compounds with at least one quaternary ammonium group can also include those compounds of formula (B) when L in formula (B) is a quaternized amino group as described.

i) amino functional silicones described as amphiphilic aminopolyorganosiloxanes and corresponding to the following formula (L):

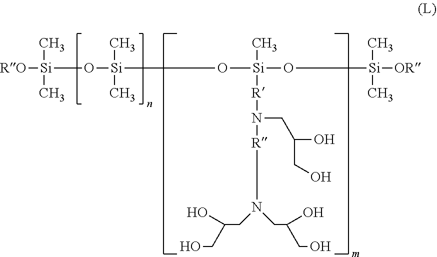

in which:

R′″, which are identical or different, denote a linear or branched C8-C20 alkyl group and more particularly a C13-C15 alkyl radical;

R′ and R″, which are identical or different, denote a linear or branched C1-C5 alkylene; n varies from 25 to 100 and preferably from 50 to 80; m varies from to 10 and preferably from 1 to 5.,

Preferably,

R′ denotes the —CH2—CH2—CH2— group or the CH2CH—CH2CH3 group,

R″ denotes —CH2—CH2—,

Use will more particularly be made of an amphiphilic aminopolyorangosiloxane corresponding to the following structure (La):

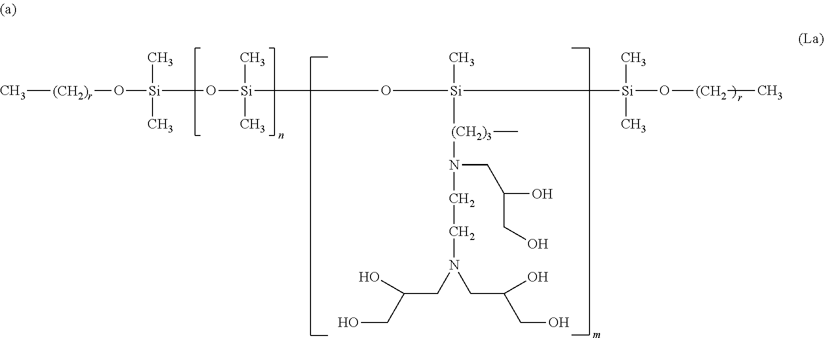

where r has a value from 12 to 14, n varies from 50 to 70 and m varies from 1 to 3, such as the product sold under the trade name “DC 8500” or Dow Corning® 8500 Conditioning Agent as sold by Dow Corning (INCI name: bis(C13-15 alkoxy) PG amodimethicone, an amino glycol copolymer).

These amphiphilic aminosilicones are known and are described in Patent Applications WO03/066007 and WO2008/011347, which are incorporated herein by reference in their entirety.

The amino functional silicone compound of the present disclosure may be provided or may be commercially available in emulsion form that further comprises surfactants chosen from nonionic surfactants, cationic surfactants, and mixtures thereof. In certain embodiments, the emulsion in which the amino functional silicone compound is contained is a microemulsion.

In some instances, the amino functional silicones in the hair conditioning compositions are chosen from amphiphilic aminosilicones, in particular, amino glycol copolymers. A sutiable example is bis(C13-15 alkoxy) PG amodimethicone.

In some instances, the at least one amino functional silicone is selected from the group consisting of bis(C13-15 alkoxy) PG amodimethicone, amodimethicone, and a mixture thereof.

The total amount of the at least one amino functional silicone in the hair conditioning compositions may vary but is typically about 0.1 to about 10 wt. %, based on the total weight of the hair conditioning composition. The total amount of the at least one amino functional silicone may be about 0.1 to about 10 wt. %, 0.1 to about 5 wt. %, about 0.5 to about 15 wt. %, about 0.5 to about 10 wt. %, about 0.5 to about 5 wt. %, about 1 to about 15 wt. %, about 1 to about 10 wt. %, about 1 to about 5 wt. %, about 1.2 to about 4 wt. %, or about 1.3 to about 3 wt. %, based on the total weight of the hair conditioning composition.

In some instances, the total amount of the at least one amino functional silicone in the hair conditioning compositions is about 0.1, 0.2, 0.3, 0.4, 0.5, 0.6, 0.7, 0.8, 0.9, 1, 1.1, 1.1.5, 1.2, 1.25, 1.3, 1.35, 1.4, 1.45, 1.5, 1.55, 1.6, 1.65, 1.7, 1.75, 1.8, 1.85, 1.9, 1.95, or 2 wt. %, based on the total weight of the hair conditioning composition.

### Nonionic Silicone Polymer

The conditioning compositions of the present disclosure contain at least one nonionic silicone polymer (or copolymer). In the context of the instant disclosure, the term “polymer” is a generic term that encompasses both “homopolymers” and “copolymers.” A high molecular weight polymer relates to a polymer having an internal phase viscosity of at least 100.000.000 cst . Additionally, the at least one nonionic silicone polymer (may have a high viscosity, for example, a viscosity of greater than 120×10⁶ mm²/s at 0.01 Hz (internal phase viscosity). The at least one nonionic silicone polymer may be present as a dispersion of particles in an appropriate carrier, for example, an aqueous carrier. Furthermore, in some instances, the nonionic silicone polymer is a silicone latex polymer.

In some instances, the nonionic silicone polymer of the present disclosure is a non-amino functional silicone polymer. In some instances, the nonionic silicone polymer according to the invention is selected from the group consisting of a polymethylsiloxane resin, a linear block copolymer (or linear block silicone copolymer), and a mixture thereof. In particular, the at least nonionic silicone polymer may be prepared from acrylic acid monomers or acrylate ester monomers. However, in some cases, the at least one nonionic silicone polymer is nonionic and is not prepared from acrylic acid monomers or acrylate ester monomers.

As noted above, the at least nonionic silicone polymer may include a polymethylsiloxane resin, which is preferably in the form of an emulsion. In certain instances, the polymethylsiloxane resin is in an aqueous emulsion medium and is present in the emulsion.

Additionally, as noted above, the at least one nonionic silicone polymer may include a linear block silicone copolymer, for example, an uncrosslinked block polymer, obtained by chain extension and not by crosslinking. The term “block copolymer” (or “sequential copolymer”) denotes a polymer comprising at least two distinct blocks (sequences). Each block of the polymer results from one type of monomer or from several types of different monomers. This means that each block can be composed of a homopolymer or of a copolymer, it being possible for this copolymer constituting the block to be in its turn a random or alternating copolymer.

The linear block silicone copolymer may include at least two distinct silicone blocks, each block of the polymer resulting from one type of silicone monomer or from several types of different silicone monomers, such as mentioned below. It should also be noted that the copolymer is “linear”; in other words, the structure of the polymer is neither branched nor star-branched nor grafted.

The linear block silicone copolymer may advantageously be in the form of particles in dispersion in an aqueous medium. The aqueous dispersion of block copolymer particles is a silicone-in-water (Sil/W) emulsion, the oily globules of which are composed of a silicone of high viscosity, so that these globules appear to form as “soft particles”. The size of the linear block silicone copolymer particles can vary widely. For example, the linear block silicone copolymer particles generally exhibit a number-average size of less than or equal to 2 microns and preferably of less than or equal to 1 micron.

The aqueous dispersions of linear block silicone copolymer particles used in the composition according to the invention can be chosen in particular from those described in EP-A-874 017, the teaching of which is incorporated here by reference in their entirety. It is possible to obtain the silicone copolymers constituting these particles by a chain extension reaction in the presence of a catalyst, starting from at least: (a) one polysiloxane (i) having at least one reactive group and preferably one or two reactive groups per molecule; and (b) one organosilicone compound (ii) which reacts with the polysiloxane (i) by a chain extension reaction.

In particular, the polysiloxane (i) is chosen from the compounds of the following formula:

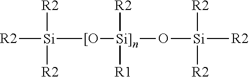

in which R1 and R2 represent, independently of one another, a hydrocarbon group having from 1 to 20 carbon atoms and preferably from 1 to 10 carbon atoms, such as methyl, ethyl, propyl or butyl, or an aryl group, such as phenyl, or a reactive group, and n is an integer greater than 1, provided that there are on average between one and two reactive groups per polymer.

The term “reactive group” is understood to mean any group capable of reacting with the organosilicone compound (ii) to form a block copolymer. Mention may be made, as reactive groups, of hydrogen; aliphatically unsaturated groups, and in particular vinyl, allyl or hexenyl groups; the hydroxyl group; alkoxy groups, such as methoxy, ethoxy or propoxy groups; alkoxy-alkoxy groups; the acetoxy group; amino groups, and mixtures thereof. Preferably, more than 90 percent and better still more than 98 percent of reactive groups are at the chain end, that is to say that the R2 radicals generally constitute more than 90 percent and even 98 percent of the reactive groups. n can in particular be an integer ranging from 2 to 100, preferably from 10 to 30 and better still from 15 to 25.

The polysiloxanes of the above formula are linear polymers, that is to say comprising few branchings and generally less than 2 mole percent of siloxane units. In some instances, it is preferable if at least 80 percent of the R1 groups are alkyl groups and better still methyl groups. Likewise, in some instances, it is preferable if the reactive group R2 at the chain end is an aliphatically unsaturated group and in particular a vinyl group. Mention may in particular be made, as polysiloxanes (i), of dimethylvinylsiloxy-polydimethylsiloxane, in which the R1 radicals are methyl radicals and the R2 radicals at the chain end are vinyl radicals while the other two R2 radicals are methyl radicals.

The organosilicone compound (ii) can be chosen from polysiloxanes of the above formula or compounds acting as chain-extending agent. If it is a compound of the above formula, the polysiloxane (i) will comprise a first reactive group and the organosilicone compound (ii) will comprise a second reactive group which will react with the first. If it is a chain-extending agent, it can be a silane, a siloxane (disiloxane or trisiloxane) or a silazane. For example, the organosilicone compound (ii) is a liquid organohydropolysiloxane of the following formula:

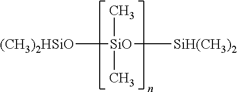

where n is an integer greater than 1 and preferably greater than 10, for example ranging from 2 to 100, preferably from 10 to 30 and better still from 15 to 25. According to a specific embodiment of the invention, n is equal to 20.

The linear block silicone copolymers may be advantageously devoid of oxyalkylene group(s), in particular devoid of oxyethylene and/or oxypropylene group(s).

In some cases, a dispersion of nonionic silicone copolymer particles is obtained from dimethylvinylsiloxy-polydimethylsiloxane (or divinyldimethicone), as compound (i), and from the compound of formula (II) with preferably n=20, as compound (ii), preferably in the presence of a catalyst of platinum type, and the dispersion of particles is preferably obtained in the presence of C₁₂-C₁₃ Pareth-3 and C₁₂-C₁₃ Pareth-23, as emulsifiers.

Use may in particular be made, as dispersion of nonionic silicone copolymer particles, of the product sold under the name HMW 2220 by Dow Corning (CTFA name: divinyldimethicone/dimethicone copolymer/C₁₂-C₁₃ Pareth-3/C₁₂-C₁₃ Pareth-23), which is a 60 percent aqueous dispersion of divinyldimethicone/dimethicone copolymer and comprising C₁₂-C₁₃ Pareth-3 and C₁₂-C₁₃ Pareth-23, said dispersion comprising about 60 percent by weight of copolymer, 2.8 percent by weight of C₁₂-C₁₃ Pareth-23, 2 percent by weight of C₁₂-C₁₃ Pareth-3 and 0.31 percent by weight of preservatives, the remainder to 100 percent being water.

The total amount of the at least one nonionic silicone polymer in the hair conditioning compositions may vary but is typically about 0.1 to about 10 wt. %, based on the total weight of the hair conditioning composition. The total amount of the at least one nonionic silicone polymer may be about 0.1 to about 10 wt. %, 0.1 to about 8 wt. %, about 0.5 to about 5 wt. %, about 0.5 to about 4 wt. %, about 0.5 to about 3 wt. %, about 1 to about 3 wt. %, or about 1 to about 2 wt. %, based on the total weight of the hair conditioning composition.

In some instances, the total amount of the at least one nonionic silicone polymer in the hair conditioning compositions is about 0.05, 0.1, 0.15, 0.2, 0.25, 0.35, 0.4, 0.45, 0.5, 0.55, 0.6, 0.65, 0.7, 0.75, 0.8, 0.85, 0.9, 0.95, 1, 1.1, 1.1.5, 1.2, 1.25, 1.3, 1.35, 1.4, 1.45, 1.5, 1.55, 1.6, 1.65, 1.7, 1.75, 1.8, 1.85, 1.9, 1.95, 2, 2.1, 2.15, 2.2, 2.25, 2.3, 2.35, 2.4, 2.45, 2.5, 2.55, 2.6, 2.65, 2.7, 2.75, 2.8, 2.85, 2.9, 2.95, or 3 wt. %, based on the total weight of the hair conditioning composition.

### Ampholytic Polymer

The hair conditioning compositions of the instant disclosure include at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type. The ampholytic polymers may include primary, secondary, tertiary, and/or quaternary amine groups that may either form part of the main polymer chain or may be borne by a side substituent directly connected thereof.

Non-limiting examples of the units derived from a monomer of (meth)acrylamide type (i) of the amphoteric polymer are units of the following structure:

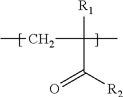

in which:

R₁ denotes H or CH₃, and

R₂ is chosen from an amino, dimethylamino, tert-butylamino or dodecylamino radical, or —NH—CH₂OH.

In some instances, the ampholytic polymer comprises a repetition of only one unit of the above formula. The unit derived from a monomer of (meth)acrylamide type of formula (I) in which R₁ denotes H and R₂ is an amino radical is particularly preferred.

It corresponds to the acrylamide monomer per se.

In some cases, the units derived from a monomer of (meth)acrylamide-alkyltrialkylammonium type (ii) of the ampholytic polymer are units of the following structure:

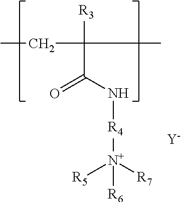

in which:

R₃ denotes H or CH₃,

R₄ denotes a group (CH₂)ₖ with k being an integer ranging from 1 to 6 and preferably from 2 to 4;

R₅ and R₆, and R₇, which may be identical or different, each denote an alkyl group containing from 1 to 4 carbon atoms; and

Y⁻ is an anion such as bromide, chloride, acetate, borate, citrate, tartrate, bisulfate, bisulfite, sulfate or phosphate.

Among these units derived from a monomer of (meth)acrylamidoalkyltrialkyl-ammonium the ones that are preferred are those derived from the methacrylamido-propyltrimethylammonium chloride monomer, which R₃ denotes a methyl radical, k is equal to 3, R₅, R₆ and R₇ denote a methyl radical, and Y⁻ denotes a chloride anion. In some cases, the ampholytic polymer comprises a repetition of only one unit of the above formula.

Finally, the units derived from an acidic monomer of (meth)acrylic acid type (iii) of the ampholytic polymer may be chosen from the units of the following structure:

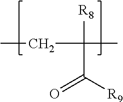

in which:

R₈ denotes H or CH₃,

R₉ denotes a hydroxyl radical or a radical —NH—C(CH₃)₂—CH₂—SO₃H. The preferred units of formula (III) correspond to the acrylic acid, methacrylic acid and 2-acrylamino-2-methylpropanesulfonic acid monomers. Preferably, the unit derived from an acidic monomer of (meth)acrylic acid type is that derived from acrylic acid, for which R₈ denotes a hydrogen atom and R₉ denotes a hydroxyl radical. The acidic monomer(s) of (meth)acrylic acid type may be non-neutralized or partially or totally neutralized with an organic or mineral base. In some cases, the amphoteric polymer comprises a repetition of only one unit of the above formula.

In some cases, the ampholytic polymer(s) comprise at least 30 mol percent of units derived from a monomer of (meth)acrylamide type. Preferably, they comprise from 30 mol percent to 70 mol percent and more preferably from 40 mol percent to 60 mol percent of units derived from a monomer of (meth)acrylamide type. The contents of units derived from a monomer of (meth)acrylamidoalkyltrialkylammonium type may advantageously be the following: from 10 mol percent to 60 mol percent and preferentially from 20 mol percent to 55 mol percent. The contents of units derived from an acidic monomer of (meth)acrylic acid type may advantageously be the following: from 1 mol percent to 20 mol percent and preferentially from 5 mol percent to 15 mol percent. Furthermore, in some caes, the amphoteric polymer comprises: from 30 mol percent to 70 mol percent and more preferably from 40 mol percent to 60 mol percent of units derived from a monomer of (meth)acrylamide type, from 10 mol percent to 60 mol percent and preferentially from 20 mol percent to 55 mol percent of units derived from a monomer of (meth)acrylamidoalkyltrialkylammonium type; from 1 mol percent to 20 mol percent and preferentially from 5 mol percent to 15 mol percent of units derived from an acidic monomer of (meth)acrylic acid type.

The ampholytic polymer(s) may also comprise additional units, other than the units derived from a monomer of (meth)acrylamide type, of (meth)acrylamidoalkyl-trialkylammonium type and of (meth)acrylic acid type. Nonetheless, in some cases, the amphoteric polymer(s) consist solely of units derived from monomers (i) of (meth)acrylamide type, (ii) of (meth)acrylamidoalkyltrialkylammonium type and (iii) of (meth)acrylic acid type.

In certain instances, the at least one ampholytic polymer may be an ampholytic terpolymer consisting of acrylic acid, methacrylamidopropyl trimethyl ammonium chloride. According to some embodiments, the ampholytic polymer is an ampholytic terpolymer known by the INCI name of polyquaternium-53 (commercially available from Nalco (Lubrizol), under the tradename, MERQUAT 2003 PR Polymer, 19.5% to 22.5% by weight active).

The at least one ampholytic polymer may have a monomer distribution such that the overall charge of the ampholytic polymer is moderately to highly cationic. For example, the ampholytic polymer may comprise about 0-20% anionic monomer, about 25-80% cationic monomer, and about 0-70% nonionic monomer. In certain iSntances, the ampholytic polymer comprises about 5-15% anionic monomer, about 30-60% cationic monomer, and about 40-60% nonionic monomer.

The cationic charge density of the ampholytic polymer may range from greater than about 1.0 meq/g to about 3.5 meq/g, such as about 1.8 meq/g to about 3.4 meq/g, about 2.0 meq/g to about 3.3 meq/g, about 2.2 meq/g to about 3.2 meq/g, about 2.3 meq/g to about 3.1 meq/g, or about 2.4 meq/g to about 3.1 meq/g. This charge density may be determined either by calculation from the structure of the polymer or experimentally via the Kjeldahl method.

Furthermore, the ampholytic polymer may have a weight average molecular weight ranging from about 500,000 to about 3,000,000, such as about 1,000,000 to about 2,000,000, or about 1,000,000 to about 1,500,000, including all ranges and subranges therebetween. In one embodiment, the ampholytic polymer has a molecular weight of about 1,000,000 to about 1,300,000 or of about 1,100,00, or of about 1,000,000, or of about 1,300,000.

The total amount of the at least one ampholytic polymer can vary but is typically about 0.01 to about 7 wt. %, based on the total weight of the hair conditioning composition. The total amount of the at least one ampholytic polymer may be about 0.01 to about 7 wt. %, about 0.01 to about 6 wt. %, about 0.01 to about 5 wt. %, about 0.05 to about 4 wt. %, about 0.1 to about 4 wt. %, about 0.1 to about 3.5 wt. %, about 0.1 to about 3 wt. %, or about 0.1 to about 1 wt. %, based on the total weight of the hair conditioning composition.

In some instances, the total amount of the at least one ampholytic polymer in the hair conditioning compositions is about 0.05, 0.1, 0.15, 0.2, 0.25, 0.35, 0.4, 0.45, 0.5, 0.55, 0.6, 0.65, 0.7, 0.75, 0.8, 0.85, 0.9, 0.95, 1, 1.1, 1.1.5, 1.2, 1.25, 1.3, 1.35, 1.4, 1.45, 1.5, 1.55, 1.6, 1.65, 1.7, 1.75, 1.8, 1.85, 1.9, 1.95, or 2 wt. %, based on the total weight of the hair conditioning composition.

### Water

The hair conditioning compositions also include water and may be in the form of an emulsion, for example, a water-in-oil emulsion, an oil-in-water emulsion, silicone-in-water emulsion, or water-in-silicone emulsion.

The total amount of water in the conditioning compositions can vary but is typically about 50 wt. % to about 95 wt. %, based on the total weight of the hair conditioning composition. The total amount of water in the conditioning compositions may be about 55 wt. % to about 95 wt. %, about 60 wt. % to about 95 wt. %, about 65 wt. % to about 95 wt. %, about 70 wt. % to about 95 wt. %, about 75 wt. % to about 95 wt. %, about 50 wt. % to about 90 wt. %, about 55 wt. % to about 90 wt. %, about 60 wt. % to about 90 wt. %, about 65 wt. % to about 90 wt. %, about 70 wt. % to about 90 wt. %, or about 75 wt. % to about 90 wt. %, based on the total weight of the hair conditioning composition.

### Cationic Surfactants

In some instances, at least one cationic surfactant is included in the conditioning compositions.

The term “cationic surfactant” means a surfactant that is positively charged when it is contained in the composition according to the disclosure. This surfactant may bear one or more positive permanent charges or may contain one or more functions that are cationizable in the composition according to the disclosure.

Non-limiting examples of cationic surfactants include behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, stearalkonium bentonite, stearalkoniumhectonite, stearyl trihydroxyethyl propylenediamine dihydrofluoride, tallowtrimonium chloride, and hexadecyltrimethyl ammonium bromide.

The cationic surfactant(s) may be chosen from optionally polyoxyalkylenated, primary, secondary or tertiary fatty amines, or salts thereof, and quaternary ammonium salts, and a mixture thereof.

The fatty amines generally comprise at least one C₈-C₃₀ hydrocarbon-based chain.

Examples of quaternary ammonium salts that may especially be mentioned include: those corresponding to the general formula (III) below:

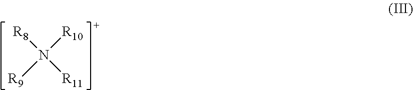

in which the groups R₈ to R₁₁, which may be identical or different, represent a linear or branched, saturated or unsaturated aliphatic group comprising from 1 to 30 carbon atoms, or an aromatic group such as aryl or alkylaryl, at least one of the groups R₈ to R₁₁ denoting a group comprising from 8 to 30 carbon atoms and preferably from 12 to 24 carbon atoms. The aliphatic groups may comprise heteroatoms especially such as oxygen, nitrogen, sulfur and halogens. The aliphatic groups are chosen, for example, from C₁-C₃₀ alkyl, C₂-C₃₀ alkenyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkyl acetate and C₁-C₃₀ hydroxyalkyl groups; X is an anion chosen from the group of halides, phosphates, acetates, lactates, (C₁-C₄)alkyl sulfates, and (C₁-C₄)alkyl- or (C₁-C₄)alkylarylsulfonates.

Among the quaternary ammonium salts of formula (III), those that are preferred are, on the one hand, tetraalkylammonium salts, for instance dialkyldimethylammonium or alkyltrimethylammonium salts in which the alkyl group contains approximately from 12 to 22 carbon atoms, in particular behenyltrimethylammonium, distearyldimethylammonium, cetyltrimethylammonium or benzyldimethylstearylammonium salts, or, on the other hand, oleocetyldimethylhydroxyethylammonium salts, palmitylamidopropyltrimethylammonium salts, stearamidopropyltrimethylammonium salts and stearamidopropyldimethylcetearylammonium salts.

In some cases it is useful to use salts such as the chloride salts of the following compounds:

A. a quaternary ammonium salt of imidazoline, such as, for example, those of formula (IV) below:

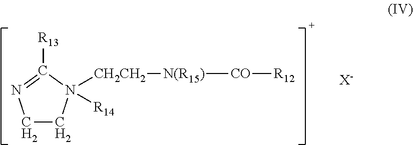

in which R₁₂ represents an alkenyl or alkyl group comprising from 8 to 30 carbon atoms, derived for example from tallow fatty acids, R₁₃ represents a hydrogen atom, a C₁-C₄ alkyl group or an alkyl or alkenyl group comprising from 8 to 30 carbon atoms, R₁₄ represents a C₁-C₄ alkyl group, R₁₅ represents a hydrogen atom or a C₁-C₄ alkyl group, X⁻ is an anion chosen from the group of halides, phosphates, acetates, lactates, alkyl sulfates, alkyl- or alkylaryl-sulfonates in which the alkyl and aryl groups preferably comprise, respectively, from 1 to 20 carbon atoms and from 6 to 30 carbon atoms. R₁₂ and R₁₃ preferably denote a mixture of alkenyl or alkyl groups containing from 12 to 21 carbon atoms, derived for example from tallow fatty acids, R₁₄ preferably denotes a methyl group, and R₁₅ preferably denotes a hydrogen atom. Such a product is sold, for example, under the name REWOQUAT W 75 by the company Evonik;

B. a quaternary diammonium or triammonium salt, in particular of formula (V):

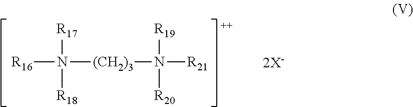

in which R₁₆ denotes an alkyl radical comprising approximately from 16 to 30 carbon atoms, which is optionally hydroxylated and/or interrupted with one or more oxygen atoms, R₁₇ is chosen from hydrogen or an alkyl radical comprising from 1 to 4 carbon atoms or a group (R₁₆ₐ)(R₁₇ₐ)(R₁₈ₐ)N—(CH₂)₃,

R₁₆ₐ, R₁₇ₐ, R₁₈ₐ, R₁₈, R₁₉, R₂₀ and R₂₁, which may be identical or different, being chosen from hydrogen and an alkyl radical comprising from 1 to 4 carbon atoms, and X⁻ is an anion chosen from the group of halides, acetates, phosphates, nitrates and methyl sulfates. Such compounds are, for example, FINQUAT CT-P, sold by the company Innospec (Quaternium 89), and FINQUAT CT, sold by the company Innospec (Quaternium 75),

C. a quaternary ammonium salt containing at least one ester function, such as those of formula (VI) below:

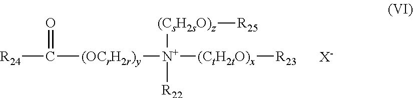

in which:

R₂₂ is chosen from C₁-C₆ alkyl groups and C₁-C₆ hydroxyalkyl or dihydroxyalkyl groups;

R₂₃ is chosen from:

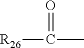

R₂₇, which is a linear or branched, saturated or unsaturated C₁-C₂₂ hydrocarbon-based group, and a hydrogen atom,

R₂₅ is chosen from:

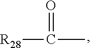

R₂₉, which is a linear or branched, saturated or unsaturated C₁-C₆ hydrocarbon-based group, and a hydrogen atom,

R₂₄, R₂₆ and R₂₈, which may be identical or different, are chosen from linear or branched, saturated or unsaturated C₇-C₂₁ hydrocarbon-based groups;

r, s and t, which may be identical or different, are integers ranging from 2 to 6;

y is an integer ranging from 1 to 10;

x and z, which may be identical or different, are integers ranging from 0 to 10;

X⁻is a simple or complex, organic or mineral anion;

with the proviso that the sum x+y+z is from 1 to 15, that when x is 0 then Rₙ denotes R₂₇, and that when z is 0 then R₂₅ denotes R₂₉.

The alkyl groups R₂₂ may be linear or branched, and more particularly linear. In some cases, R₂₂ denotes a methyl, ethyl, hydroxyethyl or dihydroxypropyl group, and more particularly a methyl or ethyl group. Advantageously, the sum x+y+z is from 1 to 10.

When R₂₃ is a hydrocarbon-based group R₂₇, it may be long and contain from 12 to 22 carbon atoms, or may be short and contain from 1 to 3 carbon atoms. When R₂₅ is an R₂₉ hydrocarbon-based group, it preferably contains 1 to 3 carbon atoms. Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

In some cases, x and z, which may be identical or different, have values of 0 or 1. Likewise, in some cases y is equal to 1. In some cases, r, s and t, which may be identical or different, are equal to 2 or 3, and even more particularly are equal to 2.

The anion X⁻ is may be a halide (chloride, bromide or iodide) or an alkyl sulfate, more particularly methyl sulfate. However, use may be made of methanesulfonate, phosphate, nitrate, tosylate, an anion derived from an organic acid, such as acetate or lactate, or any other anion compatible with the ammonium containing an ester function.

The anion X⁻ is even more particularly chloride or methyl sulfate.

Use is made more particularly, in the composition according to the invention, of the ammonium salts of formula (VI) in which:

R₂₂ denotes a methyl or ethyl group,

x and y are equal to 1;

z is equal to 0 or 1;

r, s and t are equal to 2;

R₂₃ is chosen from:

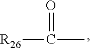

methyl, ethyl or C₁₄-C₂₂ hydrocarbon-based groups, and a hydrogen atom;

R₂₅ is chosen from:

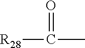

and a hydrogen atom;

R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups. The hydrocarbon-based groups are advantageously linear.

Mention may be made, for example, of the compounds of formula (VI) such as the diacyloxyethyldimethylammonium, diacylo xyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and a mixture thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with C₁₀-C₃₀ fatty acids or with mixtures of C₁₀-C₃₀ fatty acids of plant or animal origin, or by transesterification of the methyl esters thereof. This esterification is followed by quaternization using an alkylating agent such as an alkyl (preferably methyl or ethyl) halide, a dialkyl (preferably methyl or ethyl) sulfate, methyl methanesulfonate, methyl para-toluenesulfonate, glycol chlorohydrin or glycerol chlorohydrin. Such compounds are, for example, sold under the names DEHYQUART by the company BASF, STEPANQUAT by the company Stepan, NOXAMIUM by the company Ceca or REWOQUAT WE 18 by the company Evonik.

In some embodiments, the cationic surfactants that may be included in the conditioning compositions include cetrimonium chloride, cetrimonium methosulfate, stearimonium chloride, behentrimonium chloride, behentrimonium methosulfate, behenamidopropyltrimonium methosulfate, dipalmitoylethyl hydroxyethylmonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, isostearamidopropyl dimethylamine, oleyl hydroxyethyl imidazoline, stearamidopropyldimethylamine, behenamidopropyl-dimethylamine, behenamidopropyldiethylamine, behenamidoethyldiethylamine, behenamidoethyldimethylamine, arachidamidopropyldimethylamine, arachidamido-propyidiethylamine, arachidamidoethyidiethylamine, arachidamidoethyidimethylamine, quaternium-91, and a mixture thereof.

The total amount of the at least one cationic surfactant is a about 0.01 to about 10 wt. %, based on the total weight of the hair conditioning composition. The total amount of the at least one cationic surfactant may be about 0.01 to about 8 wt. %, about 0.01 to about 5 wt. %, about 0.05 to about 10 wt. %, about 0.05 to about 8 wt. %, about 0.05 to about 5 wt. %, about 0.1 to about 10 wt. %, about 0.1 to about 8 wt. %, about 0.1 to about 5 wt. %, about 1 to about 10 wt. %, about 1 to about 8 wt. %, about 1 to about 5 wt. % or about 1.5 to about 3 wt. %, based on the total weight of the hair conditioning composition.

In some instances, the total amount of the at least one cationic surfactant in the hair conditioning compositions is about 0.05, 0.1, 0.15, 0.2, 0.25, 0.35, 0.4, 0.45, 0.5, 0.55, 0.6, 0.65, 0.7, 0.75, 0.8, 0.85, 0.9, 0.95, 1, 1.1, 1.1.5, 1.2, 1.25, 1.3, 1.35, 1.4, 1.45, 1.5, 1.55, 1.6, 1.65, 1.7, 1.75, 1.77, 1.8, 1.85, 1.9, 1.95, 2, 2.1, 2, 2.25, 2.3, 2.35, 2.4, 2.45, 2.5, 2.55, 2.6, 2.65, 2.7, 2.75, 2.8, 2.85, 2.9, 2.95, or 3 wt. %, based on the total weight of the hair conditioning composition.

### Non-Silicone Fatty Compound

The hair conditioning compositions may also include at least one non-silicone fatty compound. A “fatty compound” is an organic compound that is not soluble in water at normal temperature (25° C.) and at atmospheric pressure (750 mmHg) (solubility below 10%). In some instances, the solubility in water may be below 5%, below 1%, or below 0.1%). Moreover, fatty compounds are generally soluble in one or more organic solvents under the same conditions of temperature and pressure, for example organic solvents such as chloroform, ethanol, benzene or decamethylcyclopentasiloxane. A “non-silicone fatty compound” is a fatty compound whose structure does not comprise any silicon atoms. The non-silicone fatty compounds generally have in their structure a hydrocarbon-based chain comprising at least 6 carbon atoms and not comprising any siloxane groups.

Non-limiting examples of non-silicone fatty compounds include oils, mineral oil, fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof. In some instances, the at least one non-silicone fatty compound includes one or more fatty alcohols, fatty acids, esters of fatty acids, and/or esters of fatty alcohols (for example, cetyl palmitate, cetyl stearate, myristyl myristate, myristyl stearate, cetyl myristate, and stearyl stearate (a mixture of which is referred to as “cetyl esters”)).

Non-silicone fatty compounds include hydrocarbons, fatty alcohols, fatty alcohol derivatives, fatty acids, fatty acid derivatives, fatty esters, fatty ethers, oils, waxes, etc. The non-silicone fatty compounds may be liquid or solid at room temperature and at atmospheric pressure (25° C., 1 atm). Non-limiting examples of the fatty alcohols, fatty acids, fatty alcohol derivatives, and fatty acid derivatives are found in International Cosmetic Ingredient Dictionary, Sixteenth Edition, 2016, which is incorporated by reference herein in its entirety.

Non-limiting examples of hydrocarbons include linear or branched, optionally cyclic C₆-C₁₆ alkanes; hexane, undecane, dodecane, tridecane, and isoparaffins, for instance isohexadecane, isododecane and isodecane. Additionally, the linear or branched hydrocarbons may be composed only of carbon and hydrogen atoms of mineral, plant, animal or synthetic origin with more than 16 carbon atoms, such as volatile or non-volatile liquid paraffins, petroleum jelly, liquid petroleum jelly, polydecenes, hydrogenated polyisobutene, and squalane.

The fatty alcohols that can be used may be liquid at 25° C., 1 atm, or may even be solid. They may even be glycerolated and/or oxyalkylenated, and may include from 8 to 30 carbon atoms. They may be saturated or unsaturated. The fatty alcohols useful herein include those having from about 10 to about 30 carbon atoms, from about 12 to about 22 carbon atoms, and from about 16 to about 22 carbon atoms. These fatty alcohols can be straight or branched chain alcohols and can be saturated or unsaturated. Nonlimiting examples of fatty alcohols include decyl alcohol, undecyl alcohol, dodecyl, myristyl, cetyl alcohol, stearyl alcohol, isostearyl alcohol, isocetyl alcohol, behenyl alcohol, linalool, oleyl alcohol, cholesterol, cis4-t-butylcyclohexanol, myricyl alcohol and a mixture thereof. In some cases, the fatty alcohols are those selected from the group consisting of cetyl alcohol, stearyl alcohol, isostearyl alcohol, oleyl alcohol, isotridecyl alcohol, and a mixture thereof.

The saturated liquid fatty alcohols may be branched. They can optionally comprise, in their structure, at least one aromatic or non-aromatic ring, but are preferably acyclic. Non-limiting examples of liquid saturated fatty alcohols include octyldodecanol, isostearyl alcohol, and 2-hexyldecanol.

The unsaturated liquid fatty alcohols include in their structure at least one double or triple bond and preferably one or more double bonds. When several double bonds are present, there are preferably 2 or 3 of them and they can be conjugated or non-conjugated. These unsaturated fatty alcohols can be linear or branched. They can optionally include in their structure at least one aromatic or non-aromatic ring but they are preferably acyclic. Among liquid unsaturated fatty alcohols, oleyl alcohol, linoleyl alcohol, linolenyl alcohol and undecylenyl alcohol can be cited.

Non-limiting examples of solid fatty alcohols include linear or branched, saturated or unsaturated alcohols containing from 8 to 30 carbon atoms, for example, myristic alcohol, cetyl alcohol, stearyl alcohol and their mixture, cetylstearyl alcohol.

Non-limiting examples of liquid fatty esters include esters from a C₆-C₃₂ fatty acid and/or a C₆-C₃₂ fatty alcohol, and are liquid at 25° C., 1 atm. These esters may be liquid esters of saturated or unsaturated, linear or branched C₁-C₂₆ aliphatic mono or polyacids and of saturated or unsaturated, linear or branched C₁-C₂₅ aliphatic mono or polyalcohols, the total number of carbon atoms in the esters being greater than or equal to 10. In some cases, for the esters of monoalcohols, at least one of the alcohol or the acid from which the esters of the invention result is branched. Among the monoesters of monoacids and of monoalcohols, mention may be made of ethyl palmitate, isopropyl palmitate, alkyl myristates such as isopropyl myristate or ethyl myristate, isocetyl stearate, 2-ethylhexyl isononanoate, isononyl isononanoate, isodecyl neopentanoate and isostearyl neopentanoate.

In some cases, it is particularly useful to include cetyl esters in the hair conditioning compositions. Cetyl Esters is a mixture of the following esters of saturated fatty acids and fatty alcohols: cetyl palmitate, cetyl stearate, myristyl myristate, myristyl stearate, cetyl myristate, and stearyl stearate.

Fatty alcohol derivatives include alkyl ethers of fatty alcohols, alkoxylated fatty alcohols, alkyl ethers of alkoxylated fatty alcohols, esters of fatty alcohols and a mixture thereof. Nonlimiting examples of fatty alcohol derivatives include materials such as methyl stearyl ether; 2-ethylhexyl dodecyl ether; stearyl acetate; cetyl propionate; the ceteth series of compounds such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

Esters of C₄-C₂₂ dicarboxylic or tricarboxylic acids and of C₁-C₂₂ alcohols and esters of monocarboxylic, dicarboxylic or tricarboxylic acids and of C₄-C₂₆ dihydroxy, trihydroxy, tetrahydroxy or pentahydroxy non-sugar alcohols may also be used. Mention may be made in particular of diethyl sebacate; diisopropyl sebacate; diisopropyl adipate; di-n-propyl adipate; triisopropyl citrate; glyceryl trilactate; glyceryl trioctanoate; neopentyl glycol diheptanoate; and diethylene glycol diisononanoate.

Mention may also be made of sugar esters and diesters of C₆-C₃₀ and preferably C₁₂-C₂₂ fatty acids. The term “sugar” means oxygen-bearing hydrocarbon-based compounds containing several alcohol functions, with or without aldehyde or ketone functions, and which comprise at least 4 carbon atoms. These sugars can be monosaccharides, oligosaccharides or polysaccharides. Examples of suitable sugars that may be mentioned include saccharose, glucose, galactose, ribose, fucose, maltose, fructose, mannose, arabinose, xylose and lactose, and derivatives thereof, especially alkyl derivatives, such as methyl derivatives, for instance methylglucose. The sugar esters of fatty acids may be chosen in particular from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or non-conjugated carbon-carbon double bonds. The esters according to this variant may also be chosen from mono-, di-, tri- and tetraesters, and polyesters, and mixtures thereof. These esters may be, for example, oleates, laurates, palmitates, myristates, behenates, cocoates, stearates, linoleates, linolenates, caprates and arachidonates, and mixtures thereof, such as, in particular, oleopalmitate, oleostearate or palmitostearate mixed esters. More particularly, use is made of monoesters and diesters and in particular of sucrose, glucose or methylglucose mono- or dioleates, stearates, behenates, oleopalmitates, linoleates, linolenates or oleostearates.

As oils of plant origin or synthetic triglycerides that may be used in the composition of the invention as liquid fatty esters, the following can be cited, for example, triglyceride oils of plant or synthetic origin, such as liquid fatty acid triglycerides containing from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, olive oil, rapeseed oil, coconut oil, wheatgerm oil, sweet almond oil, apricot oil, safflower oil, candlenut oil, coconut oil, camellina oil, tamanu oil, babassu oil and pracaxi oil, caprylic/capric acid triglycerides, jojoba oil, and shea butter oil.

The solid fatty acid esters and/or fatty alcohol esters that may be mentioned include solid esters obtained from C₉-C₂₆ fatty acids and from C₉-C₂₅ fatty alcohols. Among these esters, mention may be made of octyldodecyl behenate, isocetyl behenate, cetyl lactate, stearyl octanoate, octyl octanoate, cetyl octanoate, decyl oleate, myristyl stearate, octyl palmitate, octyl pelargonate, octyl stearate, alkyl myristates such as cetyl myristate, myristyl myristate or stearyl myristate, and hexyl stearate.

The liquid fatty ethers may be chosen from liquid dialkyl ethers such as dicaprylyl ether. The non-liquid fatty ethers may also be chosen from dialkyl ethers and in particular dicetyl ether and distearyl ether, alone or as a mixture.

Non-limiting examples of non-silicone waxes include carnauba wax, candelilla wax, esparto wax, paraffin wax, ozokerite, plant waxes such as olive tree wax, rice wax, hydrogenated jojoba wax or absolute flower waxes, such as the blackcurrant blossom essential wax sold by Bertin (France), or animal waxes such as beeswaxes or modified beeswaxes (cerabellina), and ceramides. Non-limiting examples of ceramides include N-linoleyldihydrosphingosine, N-oleyldihydrosphingosine, N-palmityldihydrosphingosine, N-stearyldihydrosphingosine or N-behenyldihydrosphingosine, or mixtures of these compounds.

Non-limiting olyglycerol esters of fatty acids include those of the following formula:

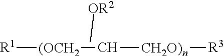

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. For example, nonionic polyglycerol esters of fatty acids include polyglyceryl-5 laurate,

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substitued fatty acids, and a mixture thereof. Nonlimiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

In some cases, the one or more fatty compounds may be one or more high melting point fatty compounds. A high melting point fatty compound is a fatty compound having a melting point of 25° C. Even higher melting point fatty compounds may also be used, for example, fatty compounds having a melting point of 40° C. or higher, 45° C. or higher, 50° C. or higher. The high melting point fatty compound may be selected from the group consisting of fatty alcohols, fatty acids, fatty alcohol derivatives, fatty acid derivatives, and mixtures thereof. Non-limiting examples of the high melting point compounds are found in International Cosmetic Ingredient Dictionary, Fifteenth Edition, 2014, which is incorporated herein by reference in its entirety. The fatty alcohols useful herein are those having from about 14 to about 30 carbon atoms, preferably from about 16 to about 22 carbon atoms. These fatty alcohols are saturated and can be straight or branched chain alcohols. Non-limiting examples of high melting point fatty compounds include fatty alcohols such as, for example, cetyl alcohol (having a melting point of about 56° C.), stearyl alcohol (having a melting point of about 58-59° C.), behenyl alcohol (having a melting point of about 71° C.), and mixtures thereof. These compounds are known to have the above melting point. However, they often have lower melting points when supplied, since such supplied products are often mixtures of fatty alcohols having alkyl chain length distribution in which the main alkyl chain is cetyl, stearyl or behenyl group. In the present application, more preferred fatty alcohols are cetyl alcohol, stearyl alcohol and mixtures thereof.

The total amount of the at least one non-silicone fatty compound may vary but is typically about 0.1 to about 20 wt. %, based on the total weight of the hair conditioning composition. The total amount of the at least one non-silicone fatty compound may be about 0.1 to about 18 wt. %, about 0.1 to about 15 wt. %, about 0.1 to about 14 wt. %, about 0.1 to about 12 wt. %, about 0.1 to about 10 wt. %, about 1 to about 9 wt. %, about 1 to about 8 wt. %, or about 2 to about 8 wt. %, based on the total weight of the hair conditioning composition.

### Nonionic Surfactants

The hair conditioning compositions may include at least one nonionic surfactant. Many nonionic surfactants are known and may be used. Nonetheless, in some cases the at least one nonionic surfactants may be selected from the group consisting of oxyethylenated fatty alcohols, block-copolymer (polycondensate) surfactants of ethylene oxide and of propylene oxide, and a mixture thereof.

The nonionic surfactant can be, for example, selected from alcohols, alpha-diols, alkylphenols and esters of fatty acids, these compounds being ethoxylated, propoxylated or glycerolated and having at least one fatty chain comprising, for example, from 8 to 18 carbon atoms, it being possible for the number of ethylene oxide or propylene oxide groups to range from 2 to 50, and for the number of glycerol groups to range from 1 to 30. Non-limiting mention may also be made of copolymers of ethylene oxide and/or of propylene oxide; condensates of ethylene oxide and/or of propylene oxide with fatty alcohols; polyethoxylated fatty amides comprising, for example, from 2 to 30 mol of ethylene oxide; polyglycerolated fatty amides comprising, for example, from 1.5 to 5 glycerol groups, such as from 1.5 to 4; ethoxylated fatty acid esters of sorbitan comprising from 2 to 30 mol of ethylene oxide; ethoxylated oils from plant origin; fatty acid esters of sucrose; fatty acid esters of polyethylene glycol; polyethoxylated fatty acid mono or diesters of glycerol (C₆-C₂₄)alkylpolyglycosides; N—(C₆-C₂₄)alkylglucamine derivatives, amine oxides such as (C₁₀-C₁₄)alkylamine oxides or N—(C₁₀-C₁₄)acylaminopropylmorpholine oxides; polyglycerolated C₈-C₄₀ alcohols, and a mixture thereof.

The nonionic surfactants may preferably be chosen from polyoxyalkylenated or polyglycerolated nonionic surfactants. The oxyalkylene units are more particularly oxyethylene or oxypropylene units, or a combination thereof, and are preferably oxyethylene units.

The surfactants preferably contain a number of moles of ethylene oxide and/or of propylene oxide of between 2 and 100 and most preferably between 2 and 50.

In accordance with one preferred embodiment of the invention, the oxyalkylenated nonionic surfactants are chosen from oxyethylenated C₈-C₃₀ alcohols.

Non-limiting examples of oxyethylenated fatty alcohols include those comprising less than 2 to about 30 OE units, preferably chosen from oxyethylenated derivatives of saturated or unsaturated, linear or branched, preferably linear, C₈-C₃₀ and preferably C₁₂-C₂₂ fatty alcohols, for instance cetyl alcohol, oleyl alcohol, oleocetyl alcohol, lauryl alcohol, behenyl alcohol, cetearyl alcohol, stearyl alcohol and isostearyl alcohol, C12-13 alcohols, and mixtures thereof. As oxyethylenated fatty alcohols comprising less than 10 OE units, mention may be made of products of addition of ethylene oxide and lauryl alcohol, for instance lauryl alcohol 2 OE (laureth-2), products of addition of ethylene oxide and stearyl alcohol, for instance stearyl alcohol 2 OE (steareth-2), products of addition of ethylene oxide and decyl alcohol, for instance decyl alcohol 3 OE (deceth-3), decyl alcohol 5 OE (deceth-5), products of addition of ethylene oxide and oleocetyl alcohol, for instance oleocetyl alcohol 5 OE (oleoceteth-5), a polyethylene glycol ether of a mixture of synthetic C12-13 fatty alcohols with an average of 3 moles of ethylene oxide (C12-13 Pareth-3), and mixtures thereof. In some instances, a polyethylene glycol ether of a mixture of synthetic C12-13 fatty alcohols with an average of 3 moles of ethylene oxide (C12-13 Pareth-3) may be particularly useful.

Furthermore, non-limiting examples of oxyethylenated fatty alcohols having an average degree of ethoxylation of 2 to 30 are, for example, laureth-2, oleth-2, ceteareth-2, laneth-2, laureth-3, oleth-3, ceteareth-3, laureth-4, oleth-4, ceteareth-4, laneth-4, laureth-5, oleth-5, ceteareth-5, laneth-5, deceth-4, deceth-7, laureth-7, oleth-7, coceth-7, ceteth-7, ceteareth-7, C11-15 pareth-7, laureth-9, oleth-9, ceteareth-9, laureth-10, oleth-10, beheneth-10, ceteareth-10, laureth-12, ceteareth-12, trideceth-12, ceteth-15, laneth-15, ceteareth-15, laneth-16, ceteth-16, oleth-16, steareth-16, oleth-20, ceteth-20, ceteareth-20, laneth-20, steareth-21, ceteareth-23, ceteareth-25, ceteareth-27, a polyethylene glycol ether of a mixture of synthetic C12-13 alcohols with an average of 23 moles of ethylene oxide (C12-13 Pareth-23), and a mixture thereof. In some instances, a polyethylene glycol ether of a mixture of synthetic C12-13 fatty alcohols with an average of 23 moles of ethylene oxide (C12-13 Pareth-3) may be particularly useful.

Furthermore, the hair conditioning composition may include one or more nonionic surfactants that is a block-copolymer (polycondensate) surfactant of ethylene oxide and of propylene oxide. The block-copolymer (polycondensate) surfactant of ethylene oxide and of propylene oxide may have a weight-average molecular weight ranging from 1000 to 20000, better from 1500 to 19000, from 2000 to 18000, or from 4000 to 17000. Mention may be made, as block-copolymer (polycondensate) surfactant of ethylene oxide and of propylene oxide which may be used, of the polyethylene glycol/polypropylene glycol/polyethylene glycol triblock polycondensates sold under the “SYNPERONIC” names, such asthose known under the INCI name of Poloxamer.

As glyceryl esters of fatty acids, glyceryl stearate (glyceryl mono-, di- and/or tristearate) (glyceryl stearate) or glyceryl ricinoleate and a mixture thereof can in particular be cited.

As glyceryl esters of C₈-C₂₄ alkoxylated fatty acids, polyethoxylated glyceryl stearate (glyceryl mono-, di- and/or tristearate) such as PEG-20 glyceryl stearate can for example be cited.

Mixtures of these surfactants, such as for example the product containing glyceryl stearate and PEG-100 stearate, marketed under the name ARLACEL 165 by Croda, and a product containing glyceryl stearate (glyceryl mono- and distearate) and potassium stearate, can also be used.

The sorbitol esters of C₈-C₂₄ fatty acids and alkoxylated derivatives thereof can be selected from sorbitan palmitate, sorbitan trioleate and esters of fatty acids and alkoxylated sorbitan containing for example from 20 to 100 EO, such as for example polyethylene sorbitan trioleate (polysorbate 85) or the compounds marketed under the trade names Tween 20 or Tween 60 by Croda.

The total amount of the at least one nonionic surfactant may vary but is typically about 0.01 to about 10 wt. %,based on the total weight of the hair conditioning composition. The total amount of the at least one nonionic surfactant may be about 0.01 to about 6 wt. %, about 0.01 to about 5 wt. %, about 0.01 to about 3 wt. %, about 0.01 to about 1 wt. %, about 0.05 to about 10 wt. %, about 0.05 to about 6 wt. %, about 0.05 to about 5 wt. %, about 0.05 to about 3 wt. %, or about 0.05 to about 1 wt. %, based on the total weight of the hair conditioning composition.

### Thickening Agents

Thickening agents that are compatible with the ingredients of the compositions may be included in the hair conditioning composition. The term “thickening agent” is interchangeable with the terms “thickener,” “gelling agent,” and “viscosity modifier.”

Many thickening agents are water-soluble, and increase the viscosity of water or form an aqueous gel when the cosmetic composition of the invention is dispersed/dissolved in water. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water. The thickening agent may include thickener polymers such as polysaccharide-based thickeners and water-soluble synthetic polymers.

Non-limiting examples of thickening agents include nonionic thickening agents such as polyacrylamides, gums, starches, cellulose and cellulose derivatives (e.g, methyl cellulose, hydroxyethylcellulose, hydroxypropylcellulose, hydroxypropyl methylcellulose, methyl hydroxyethylcellulose, microcrystalline cellulose), guar gum, hydroxypropyl guar, sclerotium gum, agarose, pectin, gellan gum,and mixtures thereof.

The total amount of the at least one thickening agent may vary but is typically about 0.01 to about 10 wt. %, based on the total weight of the hair conditioning composition. The total amount of the at least one thickening agent may be about 0.01 to about 6 wt. %, about 0.01 to about 5 wt. %, about 0.01 to about 3 wt. %, about 0.01 to about 1 wt. %, about 0.1 to about 10 wt. %, about 0.1 to about 6 wt. %, about 0.1 to about 5 wt. %, about 0.1 to about 3 wt. %, or about 0.1 to about 1 wt. %, based on the total weight of the hair conditioning composition.

### Water-Soluble Solvents

The hair conditioning composition may include at least one water-soluble solvent. The term “water-soluble solvent” is interchangeable with the term “water-miscible solvent” and means a compound that is liquid at 25 oC and at atmospheric pressure (760 mmHg), and it has a solubility of at least 50% in water under these conditions. In some cases, the water-soluble solvent has a solubility of at least 60%, 70%, 80%, or 90% in water under these conditions. Non-limiting examples of water-soluble solvents include, for example, glycerin, alcohols (for example, C1-30, C1-15, C1-10, or C1-4 alcohols), organic solvents, polyols, glycols, and a mixture thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanols (polyhydric alcohols such as glycols and polyols) such as glycerin, 1,2,6-hexanetriol, trimethylolpropane, ethylene glycol, propylene glycol, diethylene glycol, butylne glycol, hexylene glycol, triethylene glycol, tetraethylene glycol, pentaethylene glycol, dipropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

In some cases, the water-soluble solvent may be selected from the group consisting of one or more glycols, C₁₋₄ alcohols, glycerin, and a mixture thereof, for example, isopropyl alcohol, glycerin, or a mixture thereof.

The total amount of the at least one water-soluble solvent may vary but is typically about 0.1 to about 35 wt. %, based on the total weight of the hair conditioning composition. The total amount of the at least one water-soluble solvent may be about 0.1 to about 30 wt. %, about 0.1 to about 25 wt. %, about 0.1 to about 20 wt. %, about 0.1 to about 15 wt. %, about 0.1 to about 10 wt. %, about 0.1 to about 5 wt. %, about 1 to about 35 wt. %, about 1 to about 30 wt. %, about 1 to about 25 wt. %, about 1 to about 20 wt. %, about 1 to about 15 wt. %, about 1 to about 10 wt. %, or about 1 to about 5 wt. %, based on the total weight of the hair conditioning composition.

pH

The pH of the compositions according to the disclosure generally ranges from about 3 to about 6, for example, from about 3.0 to about 5.8, or from about 3.4 to about 5.5, or from about 3.5 to about 5.8, preferably from about 3.6 to about 5.5, and more preferably from about from about 3.8 to about 5.0 or such as from about 3.5 to about 4.5, including ranges and subranges therebetween. In certain embodiments, the pH of the compositions according to the disclosure is at about 3.0, 3.5, 3.6, 3.7, 3.8, 3.9, 4.0, 4.1, 4.2, 4.3, 4.4, 4.5, 4.6, 4.7, 4.8, 4.9, 5, 5.1, 5.2, 5.3, 5.4, or 5.5, and is preferably at about 3.5, 3.6, 3.7, 3.8, 3.9, 4.0, 4.1, 4.2, 4.3, 4.4 or 4.5.

### Additional Components

The composition according to the disclosure may also comprise additional components chosen from nacreous agents, colorants, fragrances, vitamins, proteins including ceramides, vitamins, UV-screening agents, free-radical scavengers, antidandruff agents, hair-loss counteractants, hair restorers, preservatives, pH stabilizers, neutralizing agents, and mixtures thereof. A person skilled in the art will take care to select the additional components (“additives”) and the amount thereof such that they do not harm the properties of the compositions of the present disclosure.

If present in the composition, these additional components are generally present in an amount ranging up to about 20% by weight of active material relative to the total weight of the composition, such as up to about 15%, up to about 12%, up to about 10%, up to about 8%, up to about 5%, such as from 0% to 20% by weight, based on the total weight of the compositions of the instant disclosure.

### Methods of Use

In an embodiment, the composition of the present disclosure is provided as part of a multi-component hair cosmetic agent for treating hair, the hair cosmetic agent comprising:

a) a first component comprising the conditioning composition of the disclosure comprising:
at least one amino functional silicone;
at least one nonionic silicone polymer;
at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
optionally, at least one cationic surfactant;
optionally, at least one non-silicone fatty compound; and water; and

b) a second component comprising a shampoo or a cleansing or an anionic surfactant-based composition;

wherein the first and second components are each packaged in a separate packaging assembly or container.

The first and second components may comprise a kit.

The compositions may be packaged in various forms, especially in bottles, in pump bottles or in tubes or in jars. The compositions may also impregnate applicators and articles, especially hair caps, gloves or wipes.

The compositions may be applied by hand, with an applicator nozzle, with a container equipped with a pump and a dispensing comb, or with an insoluble substrate or article impregnated with the composition.

In an embodiment, in accordance with a reverse-wash process, keratinous materials, such as hair, may be washed or cleansed by a first step of applying the composition of the disclosure onto hair, with an optional leave-on time, followed by a second step of applying a shampoo or cleansing or detergent-based composition, with an optional step of rinsing the hair with water between the first and second steps.

Embodiments of the disclosure also relate to a process for treating keratinous materials, such as hair, which consists in applying an effective amount of conditioning compositions of the present disclosure to the said keratinous materials, and in rinsing, for example with water, after an optional leave-on time.

Certain embodiments also relate to a process for conditioning keratinous materials such as hair, which consists in applying an effective amount of the conditioning compositions of the present disclosure to the said keratinous materials, and in optionally rinsing, for example with water, after an optional leave-on time.

The conditioning compositions may be applied to keratinous substrates, such as the hair, and subsequently rinsed off. In various embodiments, the compositions comprise hair care compositions for conditioning the hair, and in various embodiments the hair care composition will traditionally be rinsed off the hair within a short period of time after application to the hair, such as a period of time up to about 10 minutes, up to about 5 minutes, or up to about 2 minutes after application to the hair.

In various embodiments, processes according to the disclosure comprise applying the conditioning compositions described onto keratinous substrates, such as the hair, and subsequently rinsing the compositions off. The processes may, in various embodiments, impart conditioning and/or other cosmetic benefits to the keratinous substrate to which the composition is applied, even after the composition is rinsed off. The processes may additionally impart long lasting benefits such as conditioning to the keratinous substrates.

As used herein, the method/process and composition disclosed herein may be used on the hair that has not been artificially dyed, pigmented or permed.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective measurements.

Implementation of the present disclosure is provided by way of the following examples. The examples serve to illustrate the technology without being limiting in nature.

### EXAMPLES

### Example 1

### Hair Conditioning Composition

TABLE 1

| | INCI US NAME | Wt. % |
|---|---|---|
| Amino Functional | BIS(C13-15 ALKOXY) PG- | 1.5 |
| Silicone | AMODIMETHICONE (Dow | |
| | Corning 8500) | |
| Nonionic silicone | DIVINYLDIMETHICONE/ | 1.8 |
| Polymer | DIMETHICONE COPOLYMER | |
| | (Dow Corning HMW 2220) | |
| Ampholytic Polymer | POLYQUATERNIUM-53 | 0.4 |
| Cationic Surfactant | DIPALMITOYLETHYL | 1.4 |
| | HYDROXYETHYLMONIUM | |
| | METHOSULFATE, | |
| | BEHENTRIMONIUM CHLORIDE, | |
| | AND CETRIMONIUM | |
| | CHLORIDE | |
| Non-Silicone Fatty | CETYL ESTERS, CETYL | 6.2 |
| Compounds | ALCOHOL, C14-15 ALCOHOLS, | |
| | CETEARYL ALCOHOL, | |
| | ISOTRIDECYL ALCOHOL, AND | |
| | MYRISTYL ALCOHOL | |
| Water-Soluble Solvent | ISOPROPYL ALCOHOL AND | 2.1 |
| | GLYCERIN | |
| Thickening Agent | HYDROXYETHYLCELLULOSE | 0.3 |
| Nonionic Surfactant | C12-13 PARETH-3 AND | ≤1 |
| | C12-13 PARETH-23 | |
| Miscellaneous | PRESERVATIVES, PH | ≤3 |
| | ADJUSTERS, SALTS, | |
| | FRAGRANCE, ETC. | |
| Carrier | WATER | QS 100 |

### Example 2

The performance of the inventive formula in Example 1 above on hair was tested against a comparative commercial benchmark reverse wash conditioner in two separate tests—a consumer test and an expert evaluation test.

For the consumer test, the performances of both the inventive formula and comparative conditioner on hair, as used in a reverse wash routine, were compared against the performance of a consumers usual shampoo and a conditioner bundle as used in a conventional shampoo-conditioner routine (first step of applying a shampoo to hair, rinsing the hair with water, and followed by a second step of applying a conditioner to hair).

The shampoos used in the routines (regimens) mainly comprise anionic sulfate surfactants and amphoteric surfactants.

### Example 2(a)

### Consumer Testing

Consumer testing was carried out to compare the hair conditioning composition of Example 1 in a reverse wash routine with a commercial benchmark reverse wash conditioner in a home use test, wherein eleven volunteers used the innovation and benchmark products multiple times for one week per product. Each hair conditioner product was applied globally to the hair on the head of each volunteer. After the hair conditioning products were rinsed off from the hair, the hair was shampooed with a standard shampoo.

### Comparative Commercial Reverse Wash Conditioner

TABLE 2

Ingredient name
WATER (AQUA)
CETEARYL ALCOHOL
DIMETHICONE
BEHENTRIMONIUM CHLORIDE
DIPROPYLENE GLYCOL
FRAGRANCE
AMODIMETHICONE
DMDM HYDANTOIN
DISODIUM EDTA
PEG-7 PROPYLHEPTYL ETHER
CETRIMONIUM CHLORIDE
LACTIC ACID
METHYLCHLOROISOTHIAZOLINONE
ACRYLATES COPOLYMER
METHYLISOTHIAZOLINONE

The volunteers evaluated the hair treated with the reverse wash routine on a scale of 1-5 (higher values representing better results) for a variety of cosmetic characteristics relative to the volunteers' regular shampooing and conditioning routine. This method provides insight into how the tested routines (inventive routine and commercial benchmark routine) compare to each other, and also shows how the tested routines compare to the volunteers' regular routine. This information is important because consumers are not likely to switch to a routine that they do not consider an improvement over their current routine.

The results of the study are presented in the table below. If the average ranking for a particular cosmetic characteristic was less than 0.3 points different than the corresponding ranking for the individual's regular routine, the characteristic was considered equivalent to the individual's regular routine (shown as a “=” in the table below). If the average ranking for a particular characteristic was at least 0.3 points higher than the corresponding ranking for the individual's regular routine, the characteristic was considered better than the individual's regular routine (shown as a “⬆” in the table below). If the average ranking for a particular characteristic was at least 0.3 points lower than the corresponding ranking for the individual's regular routine, the characteristic was considered worse than the individual's regular routine (shown as a “⬇” in the table below).

TABLE 3

| | Comparative | Inventive |
|---|---|---|
| N = 11 | Routine | Routine |
| Wet Feel | | |
| Detangling | ↓ | = |
| Ease of Combing | ↓ | = |
| Smoothness | = | ↑ |
| Overall Wet Feel | ↓ | = |
| Dry Look and Feel | | |
| Initial Volume | = | = |
| Hair Thickness | = | = |
| Body/Bounce/Movement | = | = |
| Softness | = | = |
| Conditioned Feel | = | ↑ |
| Ease of Combing | ↓ | = |
| Hold Shape | ↓ | ↑ |
| Frizz | ↓ | = |
| Static Fly-Away | ↓ | = |
| Clean Feel | ↓ | = |
| Feels Light Weight | = | ↑ |
| Volume after 6-12 hr | ↑ | ↑ |

The data in the table above show that for every post-treatment characteristic tested, the hair conditioning composition of Example 1, when used in a reverse wash routine, was equivalent or better than the volunteer's regular routine. On the other hand, the commercial benchmark reverse wash conditioner, when used in a reverse wash routine, performed worse than the volunteer's regular routine for half of the post-treatment characteristics tested—there was only one characteristic (volume after 6-12 hr), that was ranked better than the volunteer's regular routine. Thus, the data show that hair conditioning compositions of Example 1 used in a reverse wash routine performed better than the volunteers' regular routine and better than the commercial benchmark routine. This is a significant and surprising finding.

### Example 2(b)

### Expert Evaluation

The performance/effect of the inventive conditioner on hair was evaluated in a reverse wash regimen and compared with the performance of the comparative commercial reverse wash conditioner product in Table 2 above.

The Inventive Formula and the Comparative Formula were tested on the hair of the heads of 10 human panelists in a salon by expert hair stylists. Panelists consisted of women with fine hair diameter, lightly sensitized, and medium to long hair length. Both formulas were applied and evaluated on the same person, with the Inventive Formula applied to half of the head, and the Comparative Formula applied to the other half of the head for each human panelist. After rinsing on wet hair, the Inventive Formula rated slightly higher for ease of detangling, with a tendency to also be easier to comb and pass fingers through; on dry hair, the Inventive Formula continued to trend higher for ease of combing. The amount of weight is significantly lower on wet hair for the Inventive Formula. Dry hair performance have equal ratings especially for attributes pertaining to volume and trending slightly higher for dry combing.

In summary, the inventive conditioning composition as used in a reverse wash routine provided equivalent or better performance in key conditioning attributes compared to the comparative product, even after the shampooing and rinsing steps. These results show that the conditioning effects imparted by the invention were appreciable even after the subsequent washing step. In addition, the negative effects of heavy coating or a coated feel to the hair which are imparted by conditioners (especially silicone-containing conditioners) used according to traditional or conventional routines can be minimized when the hair is treated according to the invention.

The foregoing description illustrates and describes the disclosure. Additionally, the disclosure shows and describes only the preferred embodiments but, as mentioned above, it is to be understood that the invention is capable of changes or modifications within the scope of the inventive concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” (or “comprise,” “have,” and “include”) are used in their open, non-limiting sense.

The terms “a,” “an,” and “the” are understood to encompass the plural as well as the singular.

Other than in the operating examples, or where otherwise indicated, all numbers expressing quantities may be modified in all instances by the term “about” or “approximately” meaning within +/−10% of the indicated number.

Thus, the term “a mixture thereof” also relates to “mixtures thereof.” Throughout the disclosure, if the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be include, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting.

The expression “one or more” means “at least one” and thus includes individual components as well as mixtures/combinations.

The term “rinse,” in the context of the instant disclosure, is used as customarily understood in the hair-care/hair-treatment art. For example, when a hair conditioning composition of the instant disclosure is “rinsed” from the hair, it is understood that at least some or most of the hair conditioning composition is removed from the hair. Nonetheless, in many cases, at least a residual amount of hair conditioning composition or one or more ingredient(s) of the hair conditioning composition remains in or on the hair. Typically, the residual amount of remaining composition or ingredient(s) is at least in part responsible for one or more of the styling benefits imparted to the hair.

A “rinse-off” hair-treatment composition refers to a composition that is rinsed and/or washed with water either after or during the application of the composition onto the hair, and before drying and/or styling the hair. At least a portion, and typically most, of the composition is removed from the hair during the rinsing and/or washing. The hair conditioning compositions of the instant disclosure are considered rinse-off hair conditioning compositions.

Some of the various categories of components identified for the hair conditioning compositions may overlap. In such cases where overlap may exist and the composition/product includes both components (or more than two overlapping components), an overlapping compound does not represent more than one component. For example, a fatty acid may be defined as both a “fatty compound” and may separately be considered or function as a “surfactant/emulsifier.” If a particular composition/product includes both a fatty compound and an surfactant/emulsifier, a single fatty acid can serve as only a fatty compound or a surfactant/emulsifier (a single fatty acid does not serve as both the fatty compound and the surfactant/emulsifier).

All percentages, parts and ratios herein are based upon the total weight of the compositions of the present invention, unless otherwise indicated.

“Conditioning” as used herein means imparting to one or more hair fibers at least one property chosen from combability, moisture-retentivity, luster, shine, detangling effect, frizz control, and softness. The state of conditioning can be evaluated by any means known in the art, such as, for example, measuring, and comparing, the ease of combability of the treated hair and of the untreated hair in terms of combing work (gm-in), and consumer perception.

The term “treat” (and its grammatical variations) as used herein refers to the application of the compositions of the present disclosure onto the surface of keratinous substrates such as hair. The term ‘treat,” and its grammatical variations, relates to contacting hair with the hair conditioning compositions of the present disclosure.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub-ranges between, the given ranges. Thus, a range from 1-5, includes specifically 1, 2, 3, 4 and 5, as well as sub ranges such as 2-5, 3-5, 2-3, 2-4, 1-4, etc.

The term “substantially free” or “essentially free” as used herein means that there is less than about 5% by weight of a specific material added to a composition, based on the total weight of the compositions. Nonetheless, the compositions may include less than about 3 wt. %, less than about 2 wt. %, less than about 1 wt. %, less than about 0.5 wt. %, less than about 0.1 wt. %, or none of the specified material.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

1. A hair conditioning composition comprising:
at least one amino functional silicone;
at least one nonionic silicone polymer;
at least one ampholytic polymer comprising a repetition of: (i) one or more units obtained from a monomer of (meth)acrylamide type, (ii) one or more units obtained from a monomer of (meth)acrylamidoalkyltrialkylammonium type, and (iii) one or more units obtained from an acidic monomer of (meth)acrylic acid type;
optionally, at least one cationic surfactant;
optionally, at least one non-silicone fatty compound; and
water.

2. The hair conditioning composition of claim 1, wherein the at least one amino functional silicone is selected from the group consisting of bis(C13-15 alkoxy) PG-amodimethicone, amondimethicone, and a mixture thereof.

3. The hair conditioning composition of claim 1, wherein the total amount of the amino functional silicone(s) in the hair conditioning composition is about 0.1 to about 10 wt. % based on the total weight of the hair conditioning composition.

4. The hair conditioning composition of claim 1, wherein the at least one nonionic silicone polymer is selected from the group consisting of a linear block silicone copolymer, a polymethylsiloxane resin, and mixtures thereof.

5. The hair conditioning composition of claim 4, wherein the at least one nonionic silicone polymer comprises a linear block silicone copolymer.

6. The hair conditioning composition of claim 5, wherein the linear block silicone copolymer is a divinyldimethicone/dimethicone copolymer.

7. The hair conditioning composition of claim 1, wherein the total amount of the nonionic silicone polymer(s) in the hair conditioning composition is about 0. 1 to about 10 wt. %, based on the total weight of the hair conditioning composition.

8. The hair conditioning composition of claim 1, wherein the cationic charge density of the at least one ampholytic polymer ranges from greater than 1 meq/g to about 3.5 meq/g.

9. The hair conditioning composition of claim 1, wherein the at least one ampholytic polymer comprises units derived from the following monomers: (i) acrylamide, (ii) methacrylamidopropyltrimethylammonium chloride (MAPTAC), and (iii) acrylic acid.

10. The hair conditioning composition of claim 9, wherein the at least one ampholytic polymer comprises polyquaternium-53.

11. The hair conditioning composition of claim 1, wherein the total amount of the ampholytic polymer(s) in the hair conditioning composition is about 0.01 to about 7 wt. %, based on the total weight of the hair conditioning composition.

12. The hair conditioning composition of claim 1 comprising at least one cationic surfactant.

13. The hair conditioning composition of claim 12, wherein the at least one cationic surfactant is selected from the group consisting of cetrimonium chloride, cetrimonium methosulfate, stearimonium chloride, behentrimonium chloride, behentrimonium methosulfate, behenamidopropyltrimonium methosulfate, dipalmitoylethyl hydroxyethylmonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, stearamidopropyl dimethylamine, isostearamidopropyl dimethylamine, oleyl hydroxyethyl imidazoline, stearamidopropyldimethylamine, behenamidopropyldimethylamine, behenamidopropyldiethylamine, behenamidoethyldiethyl-amine, behenamidoethyldimethylamine, arachidamidopropyldimethylamine, arachidamido-propyidiethylamine, arachidamidoethyidiethylamine, arachidamidoethyidimethylamine, quaternium-91, and a mixture thereof.

14. The hair conditioning composition of claim 13, wherein the total amount of the cationic surfactant(s) in the hair conditioning composition is about 0.01 to about 10 wt. %, based on the total weight of the hair conditioning composition.

15. The hair conditioning composition of claim 1, comprising at least one non-silicone fatty compound.

16. The hair conditioning composition of claim 15, wherein the at least one non-silicone fatty compound is selected from the group consisting of oils, mineral oil, fatty alcohols, fatty acids, fatty alcohol derivatives, fatty acid derivatives (such as alkoxylated fatty acids or polyethylene glycol esters of fatty acids or propylene glycol esters of fatty acids or butylene glycol esters of fatty acids or esters of neopentyl glycol and fatty acids or polyglycerol/glycerol esters of fatty acids or glycol diesters or diesters of ethylene glycol and fatty acids or esters of fatty acids and fatty alcohols, esters of short chain alcohols and fatty acids), esters of fatty alcohols, hydroxy-substituted fatty acids, waxes, triglyceride compounds, lanolin, and a mixture thereof.

17. The hair conditioning composition of claim 15, wherein the total amount of the non-silicone fatty compound(s) is about 0.1 to about 15 wt. %, based on the total weight of the hair conditioning composition.

18. A hair conditioning composition of claim 1 comprising:
about 0.1 to about 10 wt. %, of Bis(C13-15 alkoxy) PG-amodimethicone;
about 0.1 to about 10 wt. % of at least divinyldimethicone/dimethicone copolymer;
about 0.1 to about 5 wt. % of polyquaternium-53;
about 0.01 to about 3 wt. % of at least one cationic surfactant;
about 1 to about 15 wt. % of at least one non-silicone fatty compound; and
water.

19. A method of treating hair comprising applying a hair conditioning composition of claim 1 to the hair, and optionally rinsing the hair conditioning composition from the hair.

20. A method of treating hair comprising:
applying a hair conditioning composition of claim 1 to the hair;
optionally, rinsing the hair conditioning composition from the hair; and
after applying the hair conditioning composition to the hair and optionally rinsing the hair conditioning composition from the hair, cleansing the hair with a shampoo.

21. A kit comprising:
a hair conditioning composition of claim 1; and
a shampoo;
wherein the hair conditioning composition and the shampoo are separately contained.
